(19) United States
(12) Patent Application Publication
(10) Pub. No.: US 2026/0272621 A1
(43) Pub. Date: Sep. 17, 2026

(54) ACCURATE SCANNING OF REFLECTIVE OBJECTS

(71) Applicant: Align Technology, Inc., San Jose, CA (US)

(72) Inventor: Ofer Saphier, Rechovot (IL)

(21) Appl. No.: 19/560,947

(22) Filed: Mar. 9, 2026

Related U.S. Application Data
(60) Provisional application No. 63/770,265, filed on Mar. 11, 2025.

(51) Int. Cl. A61C 9/00; G16H 30/40

(52) U.S. Cl. CPC A61C 9/0053; G16H 30/40

(57) ABSTRACT

A method includes receiving intraoral scan data generated by an intraoral scanner, the intraoral scan data comprising one or more images of a reflective intraoral object generated during projection of light onto the reflective intraoral object by a light projector of the intraoral scanner. The method further includes identifying a virtual image of the light projector in the one or more images caused by reflection of the light off of the reflective intraoral object. The method further includes determining at least one of a position or an angle of a surface of the reflective intraoral object based at least in part on the virtual image of the light projector identified in the one or more images.

### RELATED APPLICATIONS

This patent application claims the benefit under 35 U.S.C. § 119(e) of U.S. Provisional Application No. 63/770,265, filed Mar. 11, 2025, which is incorporated by reference herein.

### TECHNICAL FIELD

Embodiments of the present disclosure relate to the field of intraoral scanning and, in particular, to a system and method for accurately capturing reflective objects during intraoral scanning.

### BACKGROUND

In prosthodontic procedures designed to insert a dental prosthesis in the oral cavity, the dental site at which the prosthesis is to be implanted in many cases should be measured accurately and studied carefully, so that a prosthesis such as a crown, denture or bridge, for example, can be properly designed and dimensioned to fit in place. A good fit enables mechanical stresses to be properly transmitted between the prosthesis and the jaw, and to prevent infection of the gums via the interface between the prosthesis and the dental site, for example.

Some intraoral objects such as highly reflective or shiny objects are particularly difficult to scan due to the reflective nature of the objects. Such objects output specular reflections when exposed to light rather than diffusing the light. This can render information of the scanned objects inaccurate and/or otherwise deficient. Such problems are particularly acute for intraoral scanners that use structured light projection.

### SUMMARY

In a first aspect of the disclosure, a method comprises: receiving intraoral scan data generated by an intraoral scanner, the intraoral scan data comprising one or more images of a reflective intraoral object generated during projection of light onto the reflective intraoral object by a light projector of the intraoral scanner; identifying a virtual image of the light projector in the one or more images caused by reflection of the light off of the reflective intraoral object; and determining at least one of a position or an angle of a surface of the reflective intraoral object based at least in part on the virtual image of the light projector identified in the one or more images.

### BRIEF DESCRIPTION OF THE DRAWINGS

The present disclosure is illustrated by way of example, and not by way of limitation, in the figures of the accompanying drawings.

FIG. 1 illustrates light reflected off of a reflective intraoral object, in accordance with embodiments of the present disclosure.

FIG. 2A illustrates a flow diagram for a method of detecting reflective intraoral objects, in accordance with embodiments of the present disclosure.

FIG. 2B illustrates a flow diagram for a further method of detecting reflective intraoral objects, in accordance with embodiments of the present disclosure.

FIG. 3A illustrates a flow diagram for a method of using a reference library to augment a representation of a reflective intraoral object, in accordance with embodiments of the present disclosure.

FIG. 3B illustrates a flow diagram for a method of determining that a reflective intraoral object corresponds to a reference object, in accordance with embodiments of the present disclosure.

FIG. 3C illustrates a flow diagram for a method of determining that a reflective intraoral object corresponds to a reference object using an AI model, in accordance with embodiments of the present disclosure.

FIG. 3D illustrates a flow diagram for a method of determining that a reflective intraoral object corresponds to a reference object, in accordance with embodiments of the present disclosure.

FIG. 4A illustrates a flow diagram for a method 400 of determining a shape of known reflective intraoral object (e.g., using information from a reference library), in accordance with embodiments of the present disclosure.

FIG. 4B illustrates a flow diagram for a method of determining a shape of an unknown reflective intraoral object, in accordance with embodiments of the present disclosure.

FIG. 5A illustrates an example virtual image of a light projector as captured in one or more images, in accordance with embodiments of the present disclosure.

FIG. 5B illustrates a virtual distance to an example virtual image of a light projector in one or more captured images, in accordance with embodiments of the present disclosure.

FIG. 5C illustrates different planes between a light projector and different estimated positions of a virtual image of the light projector in one or more captured images, in accordance with embodiments of the present disclosure.

FIG. 6 illustrates multiple possible surfaces of a reflective intraoral object, in accordance with embodiments of the present disclosure.

FIG. 7 illustrates multiple possible levels of surface smoothness of a reflective intraoral object, in accordance with embodiments of the present disclosure.

FIG. 8A illustrates view of a portion of an example dental arch with a reflective intraoral object disposed thereon, in accordance with one embodiment of the present disclosure.

FIG. 8B illustrates the view of FIG. 8A after library data has been used to augment the representation of the reflective intraoral object, in accordance with one embodiment of the present disclosure.

FIG. 9 illustrates one embodiment of a system for performing intraoral scanning and generating a virtual three dimensional model of a dental site.

FIG. 10 is a schematic illustration of a wand (e.g., intraoral scanner) with a plurality of light projectors and cameras disposed within a probe at a distal end of the wand, in accordance with embodiments of the present disclosure.

FIG. 11 is a chart depicting a plurality of different configurations for the position of the light projectors and the cameras in the probe of FIG. 10, in accordance with embodiments of the present disclosure.

FIG. 12 illustrates a block diagram of an example computing device, in accordance with embodiments of the present disclosure.

### DETAILED DESCRIPTION

Described herein is a method and apparatus for improving the quality of intraoral scans taken of dental sites that include one or more reflective or shiny objects.

When reflective objects (e.g., such as objects made out of gold, titanium, etc.) are illuminated by structured light output by an intraoral scanner, features of the structured light are reflected as one or more specular reflections rather than diffused. As a result, the projected features of the structured light that are incident on the reflective objects may not be captured by cameras of the intraoral scanner, resulting in little or no data on the reflective objects being captured during intraoral scanning. Accordingly, representations of reflective objects in three-dimensional (3D) surfaces and/or models generated from intraoral scan data may have low resolution and/or low accuracy.

One example of a reflective object that is often encountered in an oral cavity during intraoral scanning is a healing abutment. A healing abutment (also referred to as a healing cap) is an abutment used during a healing phase after a dental implant is placed in a patient's jaw. A dental implant is a surgical component that replaces the root of a missing tooth. The dental implant is generally made from titanium and is inserted into the jawbone of a patient to support artificial teeth, such as crowns, bridges, or dentures. The healing abutment is generally made out of a biocompatible material such as titanium, which is strong and durable, resistant to corrosion, easy to clean, and capable of being precisely machined. The dental implant may be designed to receive an abutment, which is a connector that attaches to the dental implant. One type of abutment is an abutment that attaches to a dental prosthetic (e.g., an artificial tooth, bridge, etc.).

After an implant is installed, a healing abutment is attached to the implant for a time period to allow the patient's mouth to heal prior to installing a dental prosthetic onto the dental implant. The healing abutment helps to maintain a shape of the gum tissue around a site of the implant to ensure proper healing and to maintain a space for the final prosthetic restoration to be attached. The healing abutment may be attached post-surgery, and may help to shape gum tissue to create an optimal contour for a final crown or bridge. The healing abutment also protects the implant from debris and bacteria during the healing process.

Healing abutments are known to be particularly difficult to scan (e.g., via intraoral scanning) due to the shape, size and materials of the healing abutments. Healing abutments have a smooth, reflective surface, which can make it difficult for the intraoral scanner to capture accurate data. The reflective surface of the healing abutment returns most of the light energy in a mirror-like fashion, and usually cannot be captured during intraoral scanning. In the rare situations where light incident on the healing abutment does get captured by a camera, the light will usually saturate an image region, reducing a quality and accuracy of the image data. Additionally, the implant itself cannot be scanned via intraoral scanning due to the implant being beneath the gingiva. However, it is important to accurately determine a position and angle at which a dental implant has been installed in a jawbone to ensure the proper fit of a dental prosthetic. As a result, special abutments called scan bodies have been developed to enable the position and orientation at which a dental implant has been installed to be accurately determined.

A scan body is a type of abutment that is used for intraoral scanning. The scan body is used in the digital workflow of implant dentistry. The scan body is designed to be attached to a dental implant, and has a material and geometry that facilitate accurate capture during intraoral scanning. Accordingly, the scan body helps in capturing the precise position and orientation of the implant in the mouth using an intraoral scanner. While the healing abutment generally extends only a few millimeters above the gingiva, the scan body may extend 10-20 mm above the gingiva. This length of the scan body helps in accurately capturing angle information of the dental implant. Accurately determining the angle of the dental implant is important, as slight differences in angle can result in large inaccuracies in implant position and/or angle. Inaccurate scanning of the object attached to the implant (e.g., healing abutment or scan body) can result in a manufactured dental prosthetic (e.g., crown or bridge) that does not fit on the patient's dental arch. Accordingly, use of scan bodies for intraoral scanning is standard.

In a traditional workflow to determine the position and orientation of a dental implant, a dentist uses a specialized tool to unscrew and remove a healing abutment from the implant. The scan body is then screwed onto the implant. The scan body is designed to fit precisely into the dental implant and to provide accurate data for a digital impression (e.g., for intraoral scanning). An intraoral scanner is used to capture the position and orientation of the scan body, which can be used to determine the position and orientation of the dental implant based on the known relative positions of the scan body and the implant. The determined position and orientation of the dental implant and/or of the scan body can then be used when designing a final prosthetic restoration. After scanning is complete, the scan body is unscrewed from the dental implant and removed. The healing abutment is then screwed back onto the implant using the specialized tool to continue protecting the dental implant and shaping the gum tissue until the dental prosthetic is ready to be installed.

The above described procedure of removing the healing abutment, installing the scan body, removing the scan body, and reinstalling the healing abutment generally takes about 15-30 minutes at a dental chair to complete. By eliminating use of the scan body, 15-30 minutes of a doctor's time can be saved per dental implant.

Embodiments described herein enable accurate intraoral scanning of reflective objects such as healing abutments. In particular, embodiments described herein enable accurate determination of the position and angle of a healing abutment, and ultimately of a dental implant, eliminating the need to use scan bodies during intraoral scanning and saving valuable time for a doctor.

In some embodiments, processing logic (e.g., of a computing device connected to an intraoral scanner) receives intraoral scan data generated by an intraoral scanner, the intraoral scan data comprising one or more images of a reflective intraoral object generated during projection of light onto the reflective intraoral object by a light projector of the intraoral scanner. The processing logic identifies a virtual image of the light projector in the one or more images caused by reflection of the light off of the reflective intraoral object. The virtual image may be identified, for example, using machine learning and/or image processing. Processing logic may determine at least one of a position or an angle of a surface of the reflective intraoral object based at least in part on the virtual image of the light projector identified in the one or more images. This may include, for example, determining a position of the virtual image in the one or more images, determining a plane that is equidistant between the light projector and the virtual image of the light projector for the one or more images, and determining at least one of the position or the angle of the surface of the reflective intraoral object based on at least one of a position or an angle of the determined plane.

Some reflective intraoral objects (e.g., some healing abutments) may have a flat or approximately flat surface. For such reflective intraoral objects, the position, angle, and shape of the surface of the reflective intraoral object may be determined with high accuracy without knowing information about the intraoral object.

For reflective intraoral objects that have more complex surface shapes, an accuracy of the position, angle, and/or shape of the reflective intraoral object may be improved by using known information about the reflective intraoral object from a library (e.g., a reference library). A practitioner (e.g., doctor or technician) may input an identity of a healing abutment or other reflective intraoral object being used, and the reflective intraoral object from the library may be registered with the detected reflective intraoral object, and the captured information about the reflective intraoral object may be augmented by adding information about the reflective intraoral object from the library. Additionally, or alternatively, the information about the reflective intraoral object may be used to improve a determination of a surface angle and/or position of a point on the surface of the reflective object.

In an example, a known reference object (e.g., reflective intraoral object) may be identified in an image of a dental site (e.g., an intraoral image generated by an intraoral scanner or an image generated by projecting a virtual 3D model of a dental site onto a 3D surface or plane) and/or in a virtual 3D model of the dental site. The image and/or virtual 3D model of the dental site may then be modified by adding additional information about the reference object to the image and/or virtual 3D model based on known properties of the reference object.

Some reflective intraoral objects may have an unknown surface. For example, gold or silver fillings in a tooth may be highly reflective, and may have irregular contours. In embodiments the position, orientation and shape of a surface of such reflective intraoral objects may be estimated in embodiments using the techniques described herein.

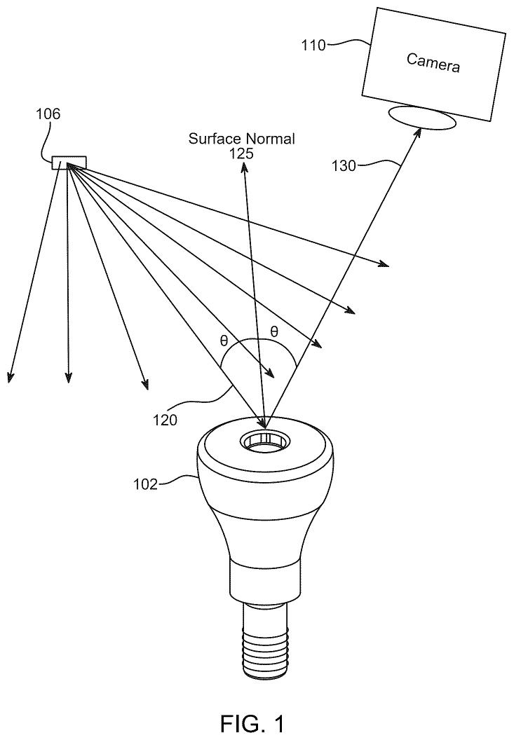

FIG. 1 illustrates light reflected off of a reflective intraoral object, in accordance with embodiments of the present disclosure. An intraoral scanner may include one or more light projectors 106 and one or more cameras 110. The light projectors 106 may be, for example, broad spectrum (e.g., white) light projectors, light projectors of a specific color or wavelength, infrared or near infrared (NIR) light projectors, and so on. The light projector(s) 106 and camera(s) 110 may have known fixed positions and orientations relative to one another. The light projector(s) 106 may project nonstructured light in some embodiments. The nonstructured light (e.g., non-patterned light) may be output from the light projector 106 at wide beam angles, with near Lambertian emission being common. In some embodiments, the light projector 106 may be covered by a lens that limits emission angle of light rays. However, even in such instances the angle of the output light rays is sufficiently wide for embodiments discussed herein to work. In some embodiments, structed light projectors are used to project structured light rather than using non-structured light projectors to project non-structured light.

The nonstructured light may include many light rays 120, each having a different angle. Because the reflective intraoral object 102 has a reflective surface, the light rays 120 that impact the reflective intraoral object 102 may experience specular reflection. Because the light includes many rays having different angles, there is a high probability that the specular reflection 130 of one of the rays of light will reach an image sensor of camera 110. In contrast, if the light were structured light, which includes many fewer rays, the probability of one of the rays of light reaching the image sensor of the camera 110 decreases. If the intraoral object had a surface that was at least partially diffuse, the light rays would diffuse when they impact the surface of the intraoral object, which enables the points of impact of the light rays with the intraoral object to be much more detectable since the light reflecting/diffusing from the surface leaves the surface at many different angles rather than at a single angle (as with specular or complete reflection).

As shown, the angle θ at which the light ray 120 hits the surface of the reflective intraoral object 102 and the angle θ of specular reflection 130 are the same. Accordingly, since the relative position and orientation of the light projector 106 and of the camera 110 are known, then the position at which the specular reflection is detected by the camera 110 (e.g., the pixel or pixels of an image sensor of camera 110 that captures the specular reflection) and the known relative positions and orientations of the light projector 106 and the camera 110 may be used to determine a surface normal 125 to the surface. The surface normal 125 may then be used to determine a plane of the surface (e.g., an angle of the plane relative to horizontal and/or relative to a longitudinal axis of the intraoral scanner) where the light ray 120 hit the surface. If the surface is flat, this same plane may apply to the entire surface (or much of the surface) of the intraoral object. Accordingly, only one or a few points need to be captured to determine the position and angle of the surface of the reflective intraoral object. In embodiments, a virtual image of the light projector may be captured in the image by the camera. The virtual image of the light projector may be used to determine the surface normal, the plane of the surface and/or the position of the surface, as explained in greater detail below.

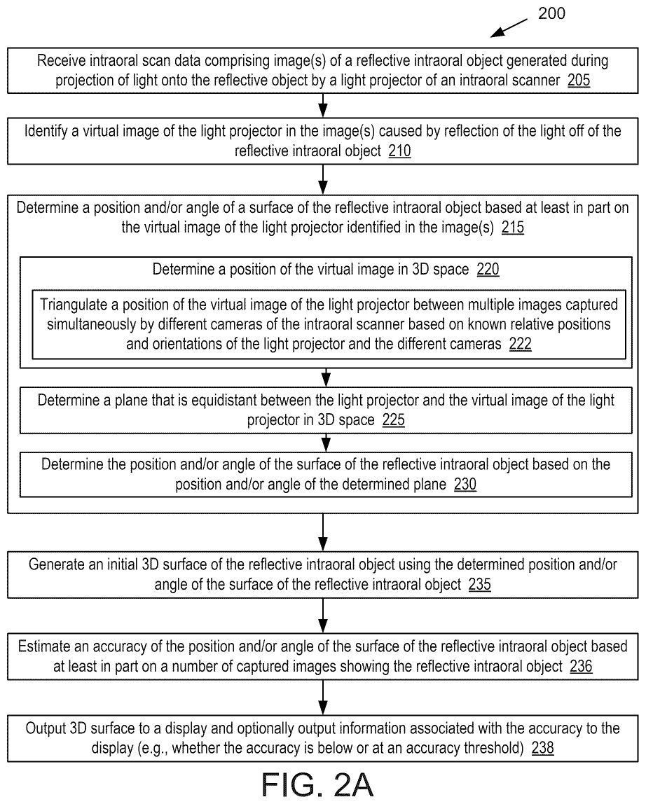

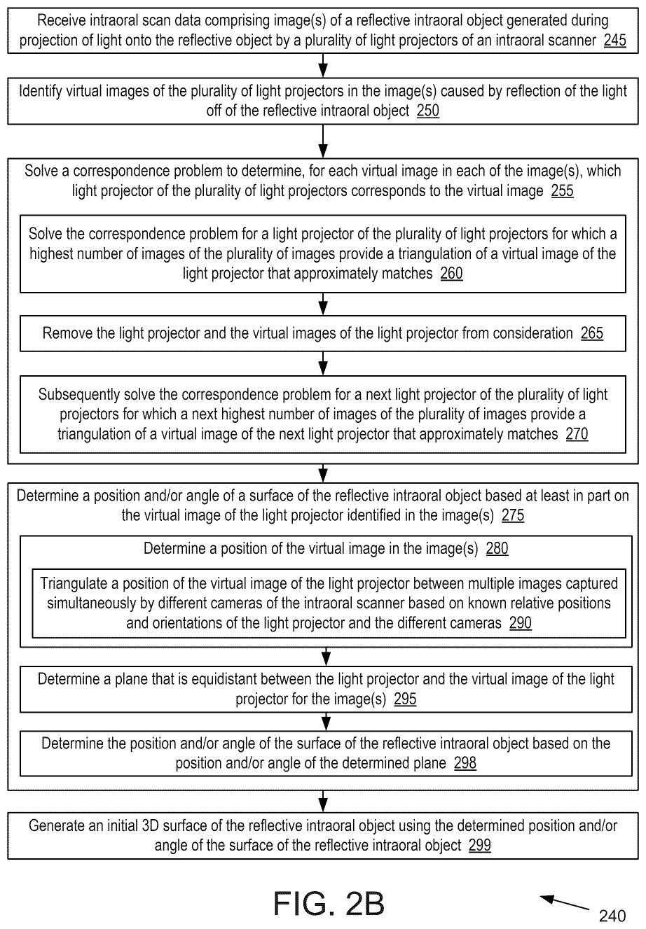

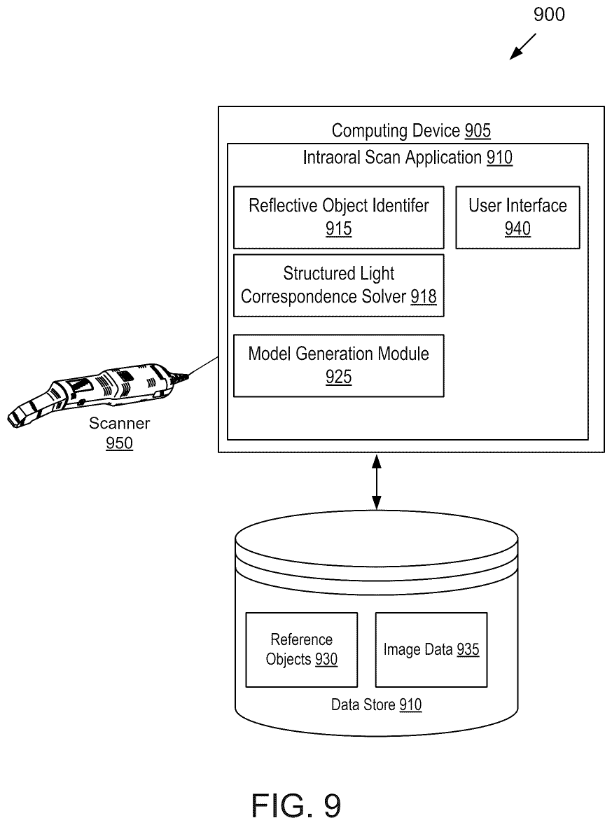

FIGS. 2A-4B illustrate methods associated with intraoral scanning as well as identification and processing of reflective intraoral objects in intraoral images generated during the intraoral scanning. The methods may be performed by processing logic that comprises hardware (e.g., circuitry, dedicated logic, programmable logic, microcode, etc.), software (such as instructions run on a processing device), or a combination thereof. Various embodiments may be performed by processing logic executing on a computing device (e.g., computing device 905 of FIG. 9) that is part of an intraoral scanning system and connected (either wirelessly or via a wired connection) to an intraoral scanner. The methods may be performed in real-time or near real-time as images are generated by a scanner and/or received from the scanner in embodiments.

FIG. 2A illustrates a flow diagram for a method 200 of detecting reflective intraoral objects, in accordance with embodiments of the present disclosure. At block 205 of method 200, processing logic receives intraoral scan data comprising images of a reflective intraoral object and optionally of surrounding non-reflective and/or at least partially diffuse intraoral objects generated during projection of light onto the reflective intraoral object and/or other intraoral objects by a light projector of an intraoral scanner. In embodiments, the light projector outputs nonstructured light, which may be used to build a surface of a reflective object in embodiments, as set forth below. Alternatively, the light projector may output structured light.

In some embodiments, the intraoral scanner alternates between projection of structured light and nonstructured light. For example, the intraoral scanner may alternate between projection of structured light for one or more frames and unstructured light for one or more frames. In such an embodiment, the structured light may be used to build a three-dimensional (3D) representation of the oral cavity of a patient, including the teeth, gingiva, palate, etc. of the patient. However, any reflective (e.g., shiny) intraoral objects in the patient's oral cavity may not be accurately captured and thus the reflective object may not be included in the 3D representation built from the structured light. For building a 3D representation of the reflective object, the operations of method 200 may be performed.

In some embodiments, the intraoral scanner includes multiple cameras (e.g., four or more cameras). Two or more of the cameras may capture (e.g., may simultaneously capture) images of the reflective object. Each of the cameras may have a known relative position and orientation relative to the light projector and relative to one another. Multiple reflections of the same light projector may also be captured in a same image in some embodiments when the surface of the reflective intraoral object is not flat. However, for a flat or slightly convex surface, at most one specular reflection of light from the light projector will be captured by each camera and represented in a respective image.

At block 210, processing logic identifies a virtual image of the light projector in the one or more images. When light from the light projector is reflected from the reflective intraoral object, a virtual image of the light projector (e.g., a reflection of the light projector) will be captured in the image. This is the same phenomenon as can be seen in a mirror. In both examples, the virtual image is an image of an object that appears to be behind the mirror but that cannot be projected onto a screen because light rays do not actually come from the image location of the virtual image. A virtual image is formed by diverging rays. The virtual image is created when light rays reflecting from the reflective object (e.g., mirror) appear to originate from a point behind the reflective object. However, these rays do not actually pass through that point. A virtual image appears to be right-side-up compared to the object (e.g., light projector) that it is a reflection of, and appears to be the same size as the object that it represents and the same distance behind the reflective object (e.g., mirror) that the object (e.g., light projector) is in front of. The virtual image is laterally inverted, and thus appears to be flipped from left to right in the captured image. Unlike real images (formed by converging rays), virtual images exist only in the perception of the observer looking into the reflective object (e.g., mirror), which in the present example is the camera(s) that capture image(s) of the reflective object.

In embodiments, the center and/or corners or edges of the virtual image of the light projector in an image captured by a camera can be estimated. The virtual image of the light projector may appear as a bright spot, which may be captured by one or more pixels of the camera. Finding the center and/or corners of the virtual image (e.g., the reflection) of the light projector in the image can be done by finding the highest value points (e.g., points having the highest intensity) in the image. In some embodiments, the determined position of the center and/or one or more corners of the virtual image of the light projector are used in determining at least of of the position or the angle of the surface of the reflective intraoral object.

In embodiments, the size of the light projector may be about 1 mm×1 mm square, or similar dimensions (e.g., 1×2 mm, 2×2 mm, 3×1 mm, 3×2 mm, 3×3 mm, etc.). The virtual image of the light projector will appear to have a same shape as the light projector possesses. For example, if the reflective object has a flat surface, then depending on an angle of the surface relative to the light projector and/or camera, the shape of the virtual image of the light projector may appear as a square or rectangle. The corners and/or center of the virtual image may be found in the image using standard image processing and/or the application of machine learning. For example, the image may be input into a trained artificial intelligence (AI) model such as a neural network, which may output coordinates of a center and/or corners of the virtual image of the light projector. The center and corners of the virtual image may be determined with high accuracy in embodiments. Accordingly, the position of the virtual image in the captured image may be determined.

At block 215, processing logic may determine a position and/or angle of a surface of the reflective object based at least in part on the virtual image of the light projector identified in the image(s). The position and/or angle of the surface of the reflective object may be determined using the identified position of the virtual reflective object and the known relative position and orientation of the light projector and the camera(s) that captured the image(s). In embodiments, the light projector is a uniform light projector (e.g., non-structured light projector), and the position and orientation of the uniform light projector relative to the camera(s) is known based on calibration. In some embodiments, the uniform light projector and cameras are calibrated as described in U.S. Publication No. 2024/0197448, published Jun. 20, 2024, which is incorporated by reference herein in its entirety.

In embodiments, the known relative position and orientation information between the light projector (e.g., uniform light projector) and camera(s) may be predetermined based on a calibration procedure of the intraoral scanner that includes the cameras and light projectors. The calibration procedure is described in U.S. Pat. No. 11,896,461, which issued Feb. 13, 2024, and which is incorporated by reference herein in its entirety. Notably, in embodiments the calibration is performed for a uniform light projector, which has historically not been done because in traditional systems the exact position and orientation of a uniform light projector relative to the cameras was not relevant. However, in embodiments the position and orientation of the uniform light projector relative to the cameras is useful for performing the operations described herein.

In some embodiments, at block 220 a position of the virtual image is determined in 3D space based on the position of the virtual image in one or more images and the known relative positions and orientations of the light projector and camera(s) that captured the images that show the virtual image of the light projector. The position in 3D space may be determined using triangulation in embodiments. When the virtual image is captured in multiple images (each by a different camera), then a position of the virtual image may be triangulated between the positions of the virtual image in each of those images in embodiments. Accordingly, in some embodiments at block 222 processing logic triangulates a position of the virtual image of the light projector between multiple images captured simultaneously by different cameras of the intraoral scanner based on known relative positions and orientations of the light projector and the different cameras, and the position at which the virtual image appears in the different images captured by the cameras. The more cameras that captured an image of the virtual image that there are, the more accurate the triangulation.

The position of the light projector may be known in 3D space. Additionally, the position of the virtual image of the light projector in 3D space may have been determined. Knowing both the virtual image position in space and the real light projector position in space, processing logic can estimate the position of the surface of the reflective object and an angle of the surface of the reflective object as a plane between the light projector and the virtual image of the light projector. Accordingly, at block 225, processing logic may determine a plane that is equidistant between the light projector and the virtual image of the light projector in 3D space. Processing logic may determine both the position of the plane and the angle of the plane. The angle of the plane may be determined relative to a coordinate system of the intraoral scanner and/or a world coordinate system of the 3D space in embodiments. At block 230, processing logic may then determine the position and/or angle of the surface of the reflective intraoral object based on the position and/or angle of the determined plane. The plane represents the surface of the reflective object. The plane will be exactly at the center between the light projector and the virtual image of the light projector in the 3D space. A normal (e.g., a vector normal) to the plane will be on a straight line connecting the light projector to the virtual image of the light projector in embodiments.

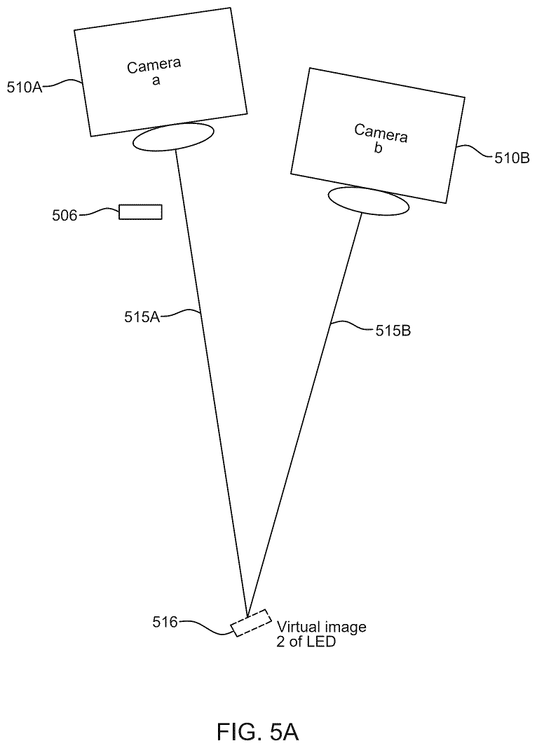

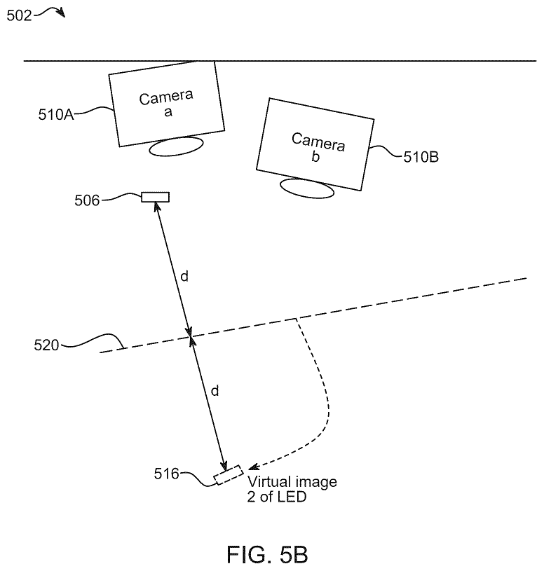

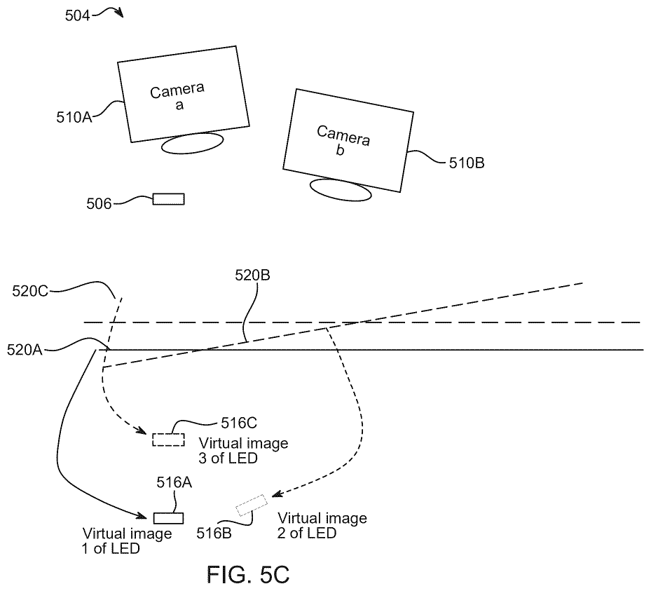

FIG. 5A illustrates an example virtual image 516 of a light projector 506 as captured in one or more images, in accordance with embodiments of the present disclosure. As shown, an intraoral scanner may include a camera 510A and a camera 510B. Each of camera 510A and camera 510B may have captured images that include virtual image 516. Rays 515A and 515B are virtual light rays between camera 510A and virtual image 516 and between camera 510B and virtual image 516, respectively.

FIG. 5B illustrates a virtual distance 525B to an example virtual image 516 of a light projector in one or more captured images, in accordance with embodiments of the present disclosure. In FIG. 5B, light projector 506, camera 510A, camera 510B, and virtual image 516 of FIG. 5A are shown. As shown, once the position of the virtual image 516 is determined in 3D space, a conceptual or virtual line may be drawn between the 3D position of the light projector 506 and the 3D position of the virtual image 516. A plane 520 may be conceptualized that is equidistant between the 3D position of the light projector 506 and the 3D position of the virtual image 516 in 3D space, where the line connecting the virtual image 516 to the light projector 506 is normal to the plane. The point at which the line between the light projector 506 and the virtual image 516 intersect the plane may correspond to a point on the 3D surface of the reflective intraoral object. As shown, a distance d between the light projector 506 and the plane 520 is equal to the distance d between the plane 520 and the virtual image 516.

FIG. 5C illustrates different planes 520A, 520B, 520C between a light projector 506 and different estimated positions of a virtual image of the light projector in one or more captured images, in accordance with embodiments of the present disclosure. As shown, when the virtual image 516A is at a first position, the plane 520A (and thus the surface of the reflective object) has a first angle and position. When the virtual image 516B is at a second position, the plane 520B (and thus the surface of the reflective object) has a second angle and position. When the virtual image 516C is at a third position, the plane 520C (and thus the surface of the reflective object) has a third angle and position.

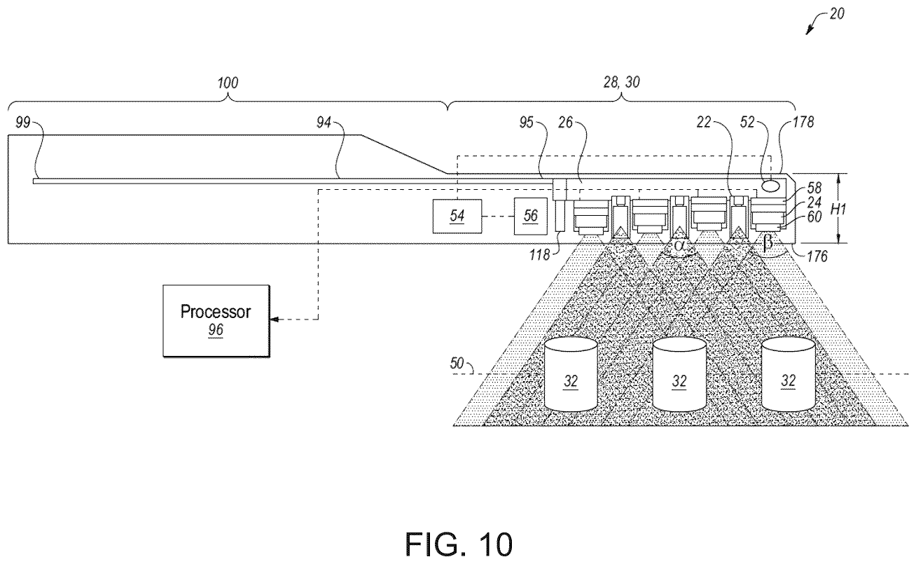

In embodiments, the virtual image position accuracy is similar to the plane position accuracy, but the plane angle accuracy is two times the virtual image position accuracy. For intraoral scanners with multiple light projectors and multiple cameras, processing logic can assume many images of the same surface from different directions. For the example intraoral scanner shown in FIG. 10, processing logic can assume that it will see the reflective object with 2-4 of the cameras of the intraoral scanner simultaneously in one embodiment. Accordingly, each virtual image is triangulated with multiple directions, which adds to the accuracy of determining the position of the virtual image.

Returning to FIG. 2A, at block 235 of method 200 processing logic generates an initial 3D surface of the reflective intraoral object using the determined position and/or angle of the surface of the reflective object. The scanner may generate a plurality of images of the reflective intraoral object from a plurality of intraoral scanner positions relative to the reflective intraoral object. Processing logic may generate the 3D surface of the reflective intraoral object using the determined position and/or angle of the surface of the reflective intraoral object from the plurality of images. If an intraoral scanner generates intraoral scans of a reflective object for two seconds, and it has a frame rate of about 10 frames per second (fps) of projection by a light projector, this gives about 2×10 fps of projection by the light projector (e.g., a broad spectrum or white light projector). This provides 10 captures of virtual images of the reflective object, and thus 10 data points for the surface of the reflective object. Additionally, the intraoral scanner may optionally project NIR light at a frame rate of 10 fps as well, providing more data points (e.g., 10 more data points for the surface of the reflective object.

As discussed above, the intraoral scanner may alternate between structured light projection and nonstructured light projection, and the nonstructured light projection may be used in embodiments to determine the shape, position and/or angle of the surface of the reflective intraoral object. In other embodiments, structured light projection may be used to determine the shape, position and/or angle of the surface of the reflective intraoral object using the same techniques that are described above. For example, some structured light projection techniques may project enough rays of light to enable the techniques described herein.

In embodiments, structured light projection is used to capture points on non-reflective or partially reflective (and partially diffuse) intraoral objects and/or non-reflective or partially reflective portions of the reflective intraoral object. The structured light may be the same light that is used to capture points on the reflective intraoral object in the above operations of method 200, or may be separate light (e.g., if the intraoral scanner alternates between projection of structured light and nonstructured light). The 3D surface of the nonreflective or partially reflective intraoral objects (or portions of the reflective intraoral object) may be determined from the structured light as described in U.S. Pat. No. 11,896,461 and/or U.S. Pat. No. 11,563,929 in embodiments.

In embodiments, the shape of the perimeter of the reflective intraoral object (e.g., the boundary between the reflective object and nonreflective objects) may be determined based on the 3D surfaces that are generated from the structured light processing. The shape of the perimeter of the reflective intraoral object may therefore be determined using the 3D surface information generated based on the structured light projection. This information may be combined with the surface information (e.g., surface plane, position, shape and/or angle) of the reflective intraoral object determined using the above described operations to generate a more accurate representation of the reflective intraoral object.

In some embodiments, some portions of the reflective intraoral object may be at least partially diffusive (e.g., may be less shiny). For such portions of the reflective intraoral object, points on the surface of the reflective intraoral object may be captured using structured light projection as described herein. Accordingly, when the surface of the reflective intraoral object (or at least parts of the surface) are both shiny and partly diffusing, processing logic collects both types of data (surface points determined based on virtual images and surface points determined based on structured light projection). In some embodiments, the reflective intraoral object as partially diffuse. In such embodiments, processing logic may determine one or more points on the surface of the reflective intraoral object based on diffused scattered light from an interaction of the light with the reflective intraoral object in the one or more images. The shape of the surface of the reflective intraoral object may be determined based on a combination of the diffused scattered light and the reflected light captured in the one or more images. For regions where the reflective intraoral object is not reflective enough to gather information based on captured virtual images of the light projector in images, only data from the structured light projection is used to determine points on the surface of the reflective intraoral object.

In some embodiments, some portions of the reflective intraoral object include surface features such as screw holes, writing, scratches, divots, protrusions, etc. These features can be triangulated using standard stereo imaging techniques (e.g., using known relative positions and orientations of a plurality of cameras that captured the features) in some embodiments. The triangulated positions of these features may be added to the 3D surface of the reflective intraoral object to improve accuracy of the 3D surface of the reflective intraoral object.

In embodiments, all positions and angles of the determined surface of the reflective intraoral object are relative to the intraoral scanner. The intraoral scanner may have a large field of view (e.g., 30, 40, 50, 60 mm, etc. in the longitudinal axis of a probe of the intraoral scanner). The large field of view of the intraoral scanner ensures that enough other regions (e.g., of non-reflective or partially reflective intraoral objects such as teeth and gingiva) are captured in images generated by the cameras of the intraoral scanner. This allows the determined surface information (e.g., 3D point cloud) of the reflective intraoral object to be registered and stitched to a 3D surface of a dental arch or portion of the dental arch that includes the other captured regions. In some embodiments, at least some of the captured images show one or more teeth in addition to the reflective intraoral object. Intraoral scan data may include one or more point clouds of at least portions of the one or more teeth determined based on structured light projection. Processing logic may stitch the one or more images to the one or more point clouds, and may determine a position and orientation of the intraoral scanner relative to the reflective intraoral object and the one or more teeth based on a result of the stitching.

In embodiments, processing logic may register the first intraoral scan to one or more additional intraoral scans using overlapping data between the various intraoral scans. In one embodiment, performing scan registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan. Each intraoral scan may be a 3D point cloud generated from multiple intraoral images captured simultaneously in embodiments.

In one embodiment, surface registration is performed for adjacent or overlapping intraoral scans (e.g., successive frames of an intraoral video). Surface registration algorithms are carried out to register two or more intraoral scans that have overlapping scan data, which essentially involves determination of the transformations which align one scan with the other. Surface registration may be performed using, for example, an iterative closest point (ICP) algorithm, and may involve identifying multiple points in multiple scans (e.g., point clouds), surface fitting to the points of each scan, and using local searches around points to match points of the overlapping scans.

Surface registration may include both stitching pairs of intraoral scans sequentially, as well as performing a global optimization that minimizes all pairs of positions together and/or or minimizes all points from all scans one to another. Accordingly, if a scan to scan registration (e.g., using ICP) searches in 6 degrees of freedom (3 translation and 3 rotation) that optimizes the distance of all points from one scan to another, then a global optimization of 11 scans will search in (11−1)×6=60 degrees of freedom for all scans relative to all other scans, while minimizing some distance between all scans. In some cases, this global optimization should give weights to different errors (e.g., edges of scans and/or far points may be given lower weight for better robustness).

Processing logic may generate a virtual 3D model of a dental object (e.g., a patient's dental arch including the reflective intraoral object and other intraoral objects) from the intraoral scans by integrating data from all intraoral scans into a single 3D model by applying the appropriate determined transformations to each of the scans. Each transformation may include rotations about one to three axes and translations within one to three planes, for example.

In one embodiment, at block 236 processing logic estimates an accuracy of the position and/or angle of the surface of the reflective object based at least in part on a number of captured images showing the reflective object. For example, the more cameras that captured the virtual image simultaneously, the greater the accuracy of the determined position in 3D space of the virtual image, and thus the greater the accuracy of the position and angle of the determined plane that represents a point on the 3D surface. Additionally, the more frames that there are in which a virtual image of the light projector was captured, the more data points there are for the surface of the reflective object, and thus the greater the accuracy of the estimated surface of the reflective object.

In one embodiment, at block 238, processing logic outputs the estimated 3D surface of the reflective object to a display. Processing logic may additionally output information associated with the accuracy of the estimated surface of the reflective object to the display. For example, processing logic may output an accuracy value to the display for the 3D surface of the reflective object. In some embodiments, processing logic compares the determined accuracy for the 3D surface of the reflective object to a threshold. If the determined accuracy is below the threshold, processing logic may output a notice to capture additional images/scans of the reflective object.

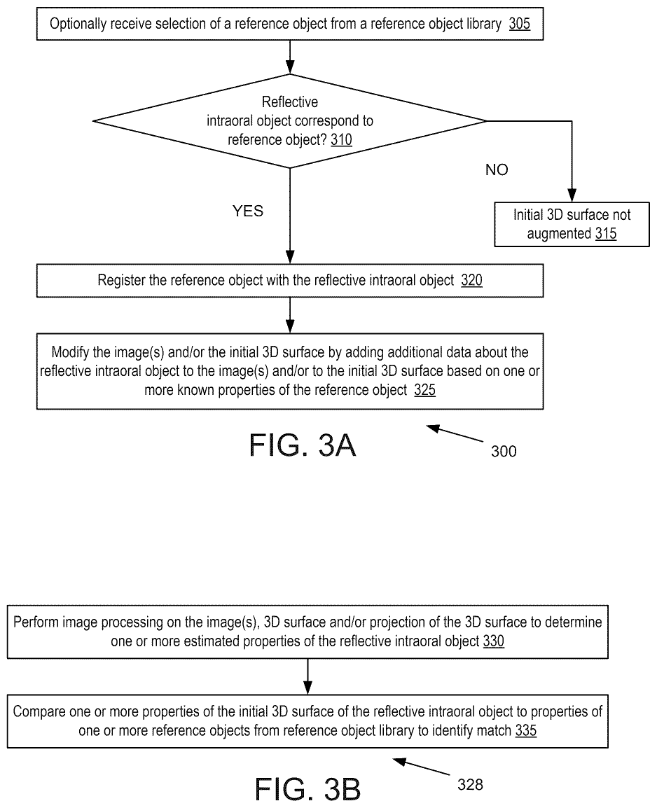

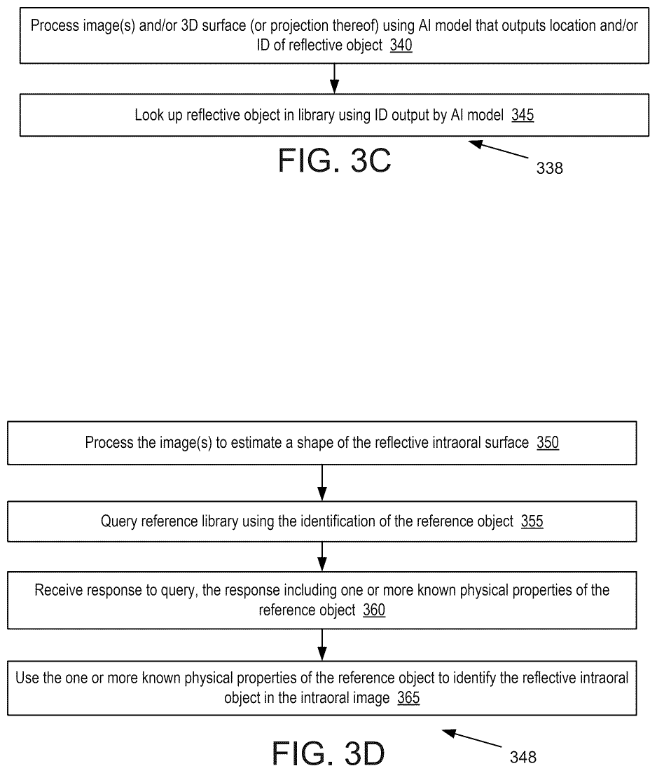

In some embodiments, the accuracy threshold may be based on known tolerances of a manufacturer of the dental implant and/or of the reflective intraoral object (e.g., healing abutment). The known tolerances and/or accuracy threshold may be added to an entry for the reflective intraoral object in the reference library in embodiments. Once the reflective intraoral object is identified (e.g., as set forth in FIGS. 3A-D), then the accuracy threshold associated with that reflective intraoral object may be retrieved and used in determining whether an achieved accuracy is sufficient.

FIG. 2B illustrates a flow diagram for a further method 240 of detecting reflective intraoral objects, in accordance with embodiments of the present disclosure. At block 245 of method 240, processing logic receives intraoral scan data comprising images of a reflective intraoral object and optionally of surrounding non-reflective and/or at least partially diffuse intraoral objects generated during projection of light onto the reflective intraoral object and/or other intraoral objects by a plurality of light projectors of an intraoral scanner. In embodiments, one or more of the light projectors outputs nonstructured light, which may be used to build a surface of the reflective object in embodiments, as set forth below.

In some embodiments, the intraoral scanner alternates between projection of structured light and nonstructured light. For example, the intraoral scanner may alternate between projection of structured light by one or more structured light projectors for one or more frames and unstructured light by the plurality of nonstructured light projectors for one or more frames.

In some embodiments, the intraoral scanner includes multiple cameras (e.g., four or more cameras). Two or more of the cameras may capture (e.g., may simultaneously capture) images of the reflective object. Each of the cameras may have a known relative position and orientation relative to the light projector and relative to one another. Multiple reflections of the same light projector(s) may also be captured in a same image in some embodiments when the surface of the reflective intraoral object is not flat. However, for a flat or slightly convex surface, at most one specular reflection of light from a particular light projector will be captured by each camera and represented in a respective image.

At block 260, processing logic identifies virtual images of the plurality of light projectors in the one or more images using the same techniques described above with reference to method 200.

Notably, it may not be immediately apparent which virtual image corresponds to which light projector. Accordingly, at block 255 processing logic may solve a correspondence problem to determine, for each virtual image in each of the images captured by the one or more cameras, which light projector of the plurality of light projectors corresponds to the virtual image.

In one embodiment, a correspondence algorithm is run to determine which virtual image in each of the images corresponds to which light projector. The images of the different cameras may have been generated at the same time and may constitute an image set in embodiments. Multiple sets of images may be generated over time, and for each set of images the correspondence problem may be solved.

Running the correspondence algorithm may include, for each set of images, determining a correspondence between the light projectors and virtual images in the set of images by determining intersections of projector rays corresponding to the light projectors with camera rays corresponding to virtual images (e.g., the positions of the virtual images in the captured images) in three-dimensional (3D) space based on calibration data that associates the camera rays corresponding to pixels on the camera sensor of each of the two or more cameras to the projector rays. Running the correspondence algorithm may include first determining the correspondence between the light projectors and the virtual images in the 3D space for a first subset of the light projectors that are associated with a highest number of virtual images in captured images. Once correspondences have been found for the first subset of the light projectors that are associated with a highest number of virtual images in captured images, processing logic may subsequently determine the correspondence between the light projectors and the virtual images in the 3D space for a second subset of the light projectors that are associated with a next highest number of virtual images. This process may be repeated, each time for light projectors associated with a next highest number of virtual images in captured images.

In one embodiment, at block 260 processing logic solves the correspondence problem for a light projector of the plurality of light projectors for which a highest number of images of the plurality of images provide a triangulation of a virtual image of the light projector that approximately matches. An approximate match may occur when virtual images from different images have the same 3D coordinates plus or minus up to a threshold allowed difference. The threshold allowed difference may be 0.1 mm, 0.2 mm, 0.3 mm, or some other difference threshold in embodiments. In one embodiment, at block 265 processing logic removes the light projector and the virtual images of the light projector from consideration. Processing logic may then subsequently solve the correspondence problem for a next light projector of the plurality of light projectors for which a next highest number of images of the plurality of images provide a triangulation of a virtual image of the next light projector that approximately match.

Processing logic may determine depths associated with virtual images based on the determined correspondence. In some embodiments, the depths may be combined with x,y information determined from the images to determine 3D coordinates of the virtual images. This information may then be used to determine 3D coordinates of points on a scanned reflective intraoral surface, as set forth in greater detail below. The depths may be determined using a correspondence algorithm and stored calibration values. The stored calibration values may associate camera rays corresponding to pixels on a camera sensor of each of a plurality of cameras to a plurality of projector rays.

Processing logic may run the correspondence algorithm using the stored calibration values in order to identify a three-dimensional location for each virtual image. In one embodiment, for a given projector ray, the processor “looks” at the corresponding camera sensor path on one of the cameras. Each detected virtual image along that camera sensor path will have a camera ray that intersects the given projector ray. That intersection defines a three-dimensional point in space. The processor may search among the camera sensor paths that correspond to that given projector ray on the other cameras and may identify how many other cameras, on their respective camera sensor paths corresponding to the given projector ray, also detected a virtual image whose camera ray intersects with that three-dimensional point in space. As used herein throughout the present application, if two or more cameras detect virtual images whose respective camera rays intersect a given projector ray at the same three-dimensional point in space, the cameras are considered to “agree” on the virtual image being located at that three-dimensional point. Accordingly, the processor may identify three-dimensional locations of the virtual image based on agreements of the two or more cameras on there being the virtual image at certain intersections. The process is repeated for the additional virtual images along a camera sensor path, and the virtual image for which the highest number of cameras “agree” is identified as the virtual image that is being projected onto the surface from the given projector ray (and thus that corresponds to a particular light projector). A three-dimensional position of the virtual image is thus computed for that virtual image, including the depth for that virtual image.

Once a position in 3D space of a virtual image is determined, the projector ray that projected that virtual image, as well as all camera rays corresponding to that virtual image, may be removed from consideration and the correspondence algorithm may be run again for a next projector ray, as described above. This may be repeated until depths are determined for many or all virtual images (or there are no remaining virtual images for which a solution can be found with a threshold level of confidence).

Ultimately, the identified three-dimensional locations may be used to determine a position and angle of a surface of the reflective intraoral object, as described below. The correspondence algorithm for solving for correspondence between virtual images and light projectors may correspond to the correspondence algorithm that is described in U.S. Pat. No. 11,896,461 with respect to finding correspondence between pattern features (e.g., corresponding to projector rays from one or more light projectors) and image features (e.g., corresponding to camera rays from one or more cameras). Additionally, the correspondence algorithm for solving for correspondence between virtual images and light projectors may correspond to the correspondence algorithm that is described in U.S. Pat. No. 11,563,929, issued Jan. 24, 2023, with respect to finding correspondence between pattern features (e.g., corresponding to projector rays from one or more light projectors) and image features (e.g., corresponding to camera rays from one or more cameras). U.S. Pat. No. 11,563,929, issued Jan. 24, 2023, is incorporated by reference herein in its entirety.

At block 275, processing logic may determine a position and/or angle of a surface of the reflective object based at least in part on the virtual images of the light projectors identified in the image(s). The position and/or angle of the surface of the reflective object may be determined using the identified positions of the virtual images of the reflective object and the known relative positions and orientations of the light projectors and the camera(s) that captured the image(s).

In some embodiments, at block 280 positions of the virtual images are determined in 3D space based on the positions of the virtual images in one or more images and the known relative positions and orientations of the light projectors and camera(s) that captured the images that show the virtual images of the light projectors. The positions in 3D space may be determined using triangulation in embodiments. When the virtual images are captured in multiple images (each by a different camera), then a position of the virtual images may be triangulated between the positions of the virtual image in each of those images in embodiments. Accordingly, in some embodiments at block 290 processing logic triangulates a position of the virtual images of the light projectors between multiple images captured simultaneously by different cameras of the intraoral scanner based on known relative positions and orientations of the light projectors and the different cameras, and the positions at which the virtual images appear in the different images captured by the cameras. The more cameras that captured an image with the virtual images that there are, the more accurate the triangulation.

The positions of the light projectors may be known in 3D space. Additionally, the positions of the virtual images of the light projector in 3D space may have been determined. Knowing both the virtual image positions in space and the real light projector positions in space, processing logic can estimate the positions (e.g., depth or distance) of the surface of the reflective object and an angle of the surface of the reflective object as a plane between the light projectors and the virtual images of the light projectors. Accordingly, at block 295, for each virtual image processing logic may determine a plane that is equidistant between the corresponding light projector and the virtual image of the light projector in 3D space. Processing logic may determine both the position of the plane and the angle of the plane. The angle of the plane may be determined relative to a coordinate system of the intraoral scanner and/or a world coordinate system of the 3D space in embodiments. At block 298, processing logic may then determine the positions and/or angles of the surface of the reflective intraoral object based on the positions and/or angles of the determined planes. The planes represent the surface of the reflective object. Each plane will be exactly at the center between a light projector and the virtual image of the light projector in the 3D space. A normal to the plane will be on a line connecting the light projector to the virtual image of the light projector.

At block 299, processing logic generates an initial 3D surface of the reflective intraoral object using the determined positions and/or angles of the surface of the reflective object. If an intraoral scanner generates intraoral scans of a reflective object for two seconds, and it has a frame rate of about 10 frames per second (fps) of projection by a light projector, this gives about 2×10 fps of projection by the light projector (e.g., a broad spectrum or white light projector). This provides 10 captures of virtual images of the reflective object, and thus 10 data points for the surface of the reflective object. Additionally, the intraoral scanner may optionally project NIR light at a frame rate of 10 fps as well, providing more data points (e.g., 10 more data points for the surface of the reflective object. If the intraoral scanner includes multiple light projectors that project light simultaneously, this further increases the number of points of the surface of the reflective object that are captured. For example, an intraoral scanner may include 6, 10 or 20 light projectors that project nonstructured light simultaneously). If we assume that the intraoral scanner has 6 light projectors that project light simultaneously, this provides 20×6=120 surface position and angle estimations for the surface of the reflective object. In some embodiments, these results may be averaged. Averaging these results will give an accurate surface position and angle for the surface of the reflective object.

In embodiments, processing logic may also perform operations 236 and 238 of method 200.

As discussed above, the intraoral scanner may alternate between structured light projection and nonstructured light projection, and the nonstructured light projection may be used in embodiments to determine the shape, position and/or angle of the surface of the reflective intraoral object. In other embodiments, structured light projection may be used to determine the shape, position and/or angle of the surface of the reflective intraoral object using the same techniques that are described above. For example, some structured light projection techniques may project enough rays of light to enable the techniques described herein.

In embodiments, structured light projection is used to capture points on non-reflective or partially reflective (and partially diffuse) intraoral objects and/or non-reflective or partially reflective portions of the reflective intraoral object. The structured light may be the same light that is used to capture points on the reflective intraoral object in the above operations of method 200, or may be separate light (e.g., if the intraoral scanner alternates between projection of structured light and nonstructured light). The 3D surface of the nonreflective or partially reflective intraoral objects (or portions of the reflective intraoral object) may be determined from the structured light as described in U.S. Pat. Nos. 11,896,461 and/or 11,563,929 in embodiments.

In embodiments, the shape of the perimeter of the reflective intraoral object (e.g., the boundary between the reflective object and nonreflective objects) may be determined based on the 3D surfaces that are generated from the structured light processing. The shape of the perimeter of the reflective intraoral object may therefore be determined using the 3D surface information generated based on the structured light projection. This information may be combined with the surface information (e.g., surface plane, position, shape and/or angle) of the reflective intraoral object determined using the above described operations to generate a more accurate representation of the reflective intraoral object.

In embodiments, methods 200 and 240 provide an initial 3D surface of a reflective intraoral object. For some types of reflective intraoral objects, such as those that have a flat surface, the number of data points that are collected of the 3D surface of the reflective object are sufficient to generate a high accuracy determination of the position and shape of the reflective object and of an angle of the reflective object. In the case of a healing abutment, the healing abutment is screwed into a dental implant. Based on the position and angle/orientation of the healing abutment, a position (e.g., depth in the jaw) and angle of the dental implant may be determined. These properties are important to accurately design a dental prosthetic to be attached to the dental implant.

For reflective intraoral objects that have a more complex surface shape (e.g., other than flat or slightly convex), the number of data points that are determined for the surface of the reflective intraoral object may be insufficient to determine the position, shape and/or angle of the reflective intraoral object with high accuracy. For such instances, methods associated with FIGS. 3A-3C may be performed to improve an accuracy of the position, shape and/or orientation of the reflective intraoral object in a 3D surface or 3D model.

FIG. 3A illustrates a flow diagram for a method 300 of using a reference library to augment a representation of a reflective intraoral object, in accordance with embodiments of the present disclosure. Method 300 may be performed after operation of block 235, 236 or 238 of method 200 and/or after operation 299 of method 240 in embodiments to generate a 3D model of the reflective intraoral object having increased accuracy.

At block 305 of method 300, processing logic optionally receives a selection of a reference object from a reference object library. The selected reference object may correspond to the reflective intraoral object. For example, the reflective intraoral object may be a healing abutment that has been screwed into a dental implant of a patient. A doctor or other dental professional may provide input selecting the reference object from a library of reference objects. The library may contain information on multiple different reference objects. The information may include materials of the reference objects, a 3D shape of the reference objects (e.g., a 3D model of the reference objects), a manufacturer of the reference objects, a model number of the reference objects, and so on.

At block 310, processing logic may determine whether the reflective intraoral object corresponds to a reference object. If the reflective intraoral object corresponds to a reference object from the reference library, the method continues to block 320. Otherwise, the method proceeds to block 315 and the initial 3D surface of the reflective intraoral object may not be augmented based on data from the reference library.

Multiple different techniques may be used to determine whether the reflective intraoral object corresponds to a reference object from the reference library. If a selection of a particular reference object was received, then processing logic may compare determined properties of the reflective intraoral object (as determined from the captured image data and/or generated 3D surface) to known properties of the reference object (e.g., the exact dimensions of the reference object). A match may be found when the determined 3D surface of the reflective intraoral object matches a known 3D surface of the reference object to within a target degree of accuracy.

In some instances, processing logic may process one or more of the images, the generated 3D surface of the reflective intraoral object, and/or a projection of the generated 3D surface of the reflective intraoral object using a trained AI model to determine an identification of the reflective intraoral object. The AI model may output, for example, a location of the reflective intraoral object, a shape of the reflective intraoral object, an identification of the reflective intraoral object (e.g., of a model number for the reflective intraoral object), and so on. In some embodiment, the AI model outputs a bounding box around the reflective intraoral object. In some embodiments, the AI model outputs segmentation information for the reflective intraoral object, which may include semantic segmentation information or instance segmentation information. In some embodiments, the AI model is an artificial neural network, such as a convolutional neural network (CNN). Other types of machine learning models and/or other AI models may also be used. Once an identifier (ID) such as a model number of the reflective intraoral object is determined, it may be used to look up information about the reference object.

In some instances, processing logic compares the determined 3D surface of the reflective intraoral object to 3D models of multiple reference objects in the reference object library until a match is found.

In some cases, a doctor may mistakenly select a wrong reference object. If a match is not found between the reflective intraoral object and a selected reference object, then processing logic may perform further processing to determine whether any other reference object from the reference library matches the reflective intraoral object. If an object type (e.g., an abutment type) is identified that is different from one selected by the doctor (or other user, such as in an Rx form), a warning can be output in embodiments.

At block 320, processing logic registers the reference object from the reference library to the 3D surface of the reflective intraoral object (and in general to a 3D model or 3D surface of a dental arch that contains the 3D surface of the reflective intraoral object). Known data from the 3D model of the reference object may then be stitched to the 3D model or 3D surface of the reflective intraoral object (and in general to the 3D model of the dental arch or portion thereof) based on the registration.

Registration may be performed using overlapping data between the generated 3D surface of the reflective intraoral object and the known 3D surface of the reference object. In one embodiment, performing scan registration includes capturing 3D data of various points of a surface of the reflective intraoral object and the reference object, and registering the reflective intraoral object 3D surface to the reference object 3D surface by computing transformations therebetween. The two 3D surfaces may then be integrated into a common reference frame by applying appropriate transformations to points in the reference object 3D surface.

Surface registration in embodiments essentially involves determination of the transformations which align one 3D surface with the other. Surface registration may be performed using, for example, an iterative closest point (ICP) algorithm, and may involve identifying multiple points in the two 3D surfaces (e.g., point clouds), surface fitting to the points of each 3D surface, and using local searches around points to match points of the overlapping 3D surfaces. In one embodiment, registration (and stitching) is performed as described in U.S. Pat. No. 6,542,249, issued Apr. 1, 2003, entitled “Three-dimensional Measurement Method and Apparatus,” which is incorporated by reference herein. Other registration techniques may also be used.

A special condition may arise when features (e.g., lines or points) that are less than a surface are to be registered to a surface. This may occur, for example, when the 3D surface of the reflective intraoral object is incomplete (e.g., due to limited data points). In the ICP, points from one surface to another are minimized, but the point correspondence step of the ICP can change in each iteration. In a variant algorithm, a fixed correspondence may be found between the feature point (e.g., of a feature of a surface) and the surface points (e.g., of a surface), and try to minimize it together with all the surface minimization. As the feature may be a single point or a few points, and may be overwhelmed by the majority of surface points, the error of this feature point will receive a high weight in the global error.

At block 325, processing logic may modify the images and/or the initial 3D surface by adding additional data about the reflective intraoral object to the image(s) and/or to the initial 3D surface based on one or more known properties (e.g., 3D dimensions) of the reference object. Processing logic may generate a virtual 3D model of a dental object (e.g., a patient's dental arch that includes the reflective intraoral object) from the by integrating data from the initial 3D surface and the 3D model of the reference object. This may include applying the appropriate determined transformations to align the 3D model of the reference object to the 3D surface or model of the reflective intraoral object on the dental arch in embodiments. Each transformation may include rotations about one to three axes and translations within one to three planes, for example.

FIG. 3B illustrates a flow diagram for a method 328 of determining that a reflective intraoral object corresponds to a reference object, in accordance with embodiments of the present disclosure. In embodiments, method 328 is performed at block 310 of method 300. At block 330 of method 328, processing logic performs image processing of one or more images, a 3D surface of a reflective intraoral object, or a projection of the 3D surface to determine one or more estimated properties of the reflective intraoral object. The properties may include, for example, a shape, a color, a texture, a reflectivity, a surface pattern, a diffraction, a reactivity to a wavelength of light, and so on. At block 335, processing logic may compare the one or more determined properties of the reflective intraoral object to properties of one or more reference objects from the reference library to identify a match.

FIG. 3C illustrates a flow diagram for a method 338 of determining that a reflective intraoral object corresponds to a reference object using an AI model, in accordance with embodiments of the present disclosure. In embodiments, method 338 is performed at block 310 of method 300. At block 340 of method 328, processing logic inputs one or more images, a 3D surface of a reflective intraoral object, or a projection of the 3D surface into a trained AI model. The trained AI model outputs one or more estimated properties of the reflective intraoral object, a location of the reflective intraoral object and/or an ID of the reflective intraoral object (e.g., a model number of an abutment). At block 345, processing logic may look up the reflective object in a library using the ID output by the AI model. Properties of the reflective intraoral object may then be compared to properties of the reflective object in the library as set forth in block 335 of method 328 in embodiments to identify a match.

FIG. 3D illustrates a flow diagram for a method 348 of determining that a reflective intraoral object corresponds to a reference object, in accordance with embodiments of the present disclosure. In embodiments, method 348 is performed at block 310 of method 300. At block 350 of method 348, processing logic processes the image(s) and/or 3D surface of the reflective intraoral object to estimate a shape of the reflective intraoral object and/or other properties of the reflective intraoral object (e.g., color, surface pattern, texture, reflectivity, diffusivity, etc.). At block 355, processing logic queries a reference library using a received identification of the reference object. At block 360, processing logic receives a response to the query, the response including one or more known physical properties of the reference object. At block 365, processing logic uses the one or more known physical properties of the reference object to identify the reflective intraoral object in the intraoral image and/or 3D surface.

In some embodiments, the reflective intraoral object may not have an entry in the reference library. In such instances, a doctor may manually add an entry for the reflective intraoral object to the library. The reference objects in the library may include well known equipment that is standard in dentist offices and/or orthodontist offices. Such reference objects may be default reference objects that a data store is prepopulated with. Some reference objects may be custom reference objects in the reference library may have been added by a dental practitioner using a training mode. Each entry for a reference object may include one or more physical properties of that reference object, such as a shape (e.g., a 3D virtual model of the reference object), color, texture, surface pattern, and so on. In one embodiment a user may cause an intraoral scan application to enter a training mode. The user (e.g., doctor) may coat the reflective intraoral object (which is not installed in a patient's mouth), in a coating such as a powder that will cause the surface of the reflective intraoral object to exhibit diffused reflectance. During the training mode, a user may use an intraoral scanner to perform a 3D scan of the reflective object with the coating. Images of the reflective object may then be used to generate a virtual 3D model of the reflective object. An entry for the scanned reflective object may then be added to the reference library. The entry may include the generated virtual 3D model, a name of the reflective object and/or one or more physical properties of the object.

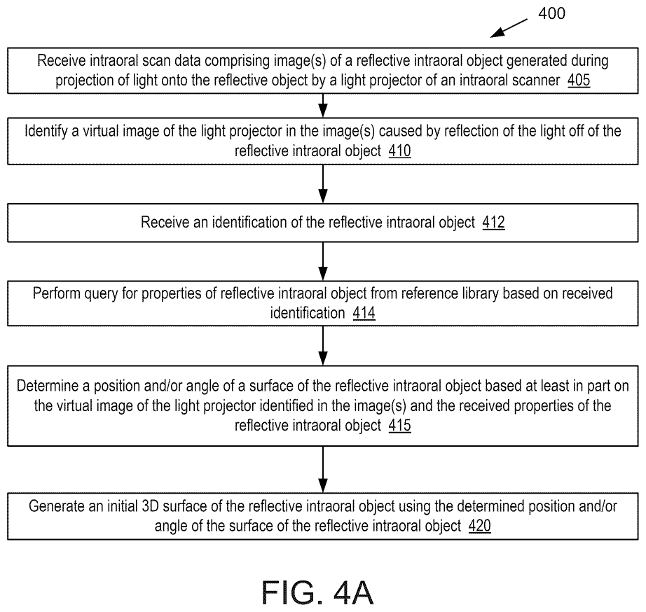

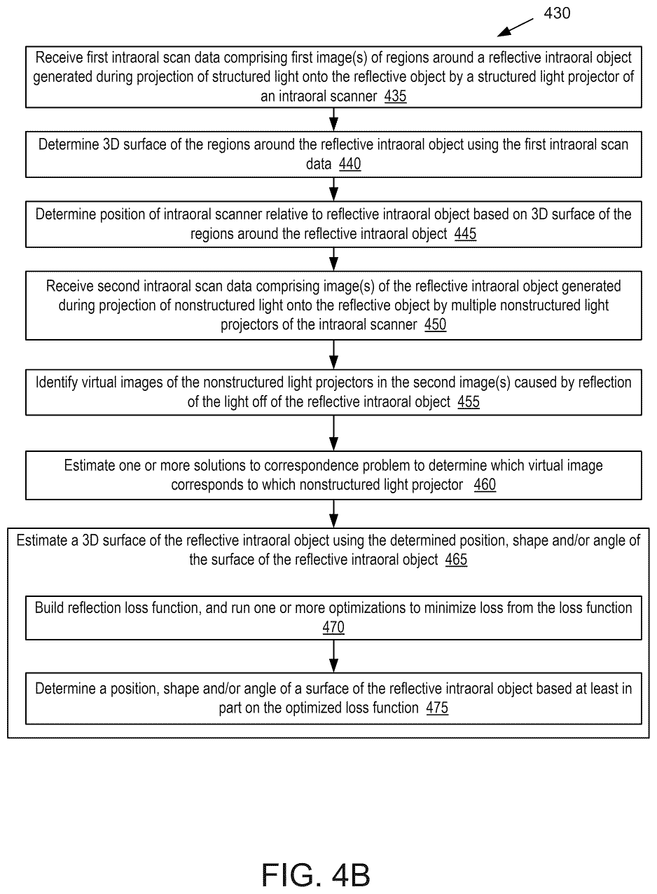

FIG. 4A illustrates a flow diagram for a method 400 of determining a shape of known reflective intraoral object (e.g., using information from a reference library), in accordance with embodiments of the present disclosure. At block 405 of method 400, processing logic receives intraoral scan data comprising images of a reflective intraoral object and optionally of surrounding non-reflective and/or at least partially diffuse intraoral objects generated during projection of light onto the reflective intraoral object and/or other intraoral objects by a light projector of an intraoral scanner. In embodiments, the light projector outputs nonstructured light, which may be used to build a surface of a reflective object in embodiments, as set forth below.

In some embodiments, the intraoral scanner alternates between projection of structured light and nonstructured light. For example, the intraoral scanner may alternate between projection of structured light for one or more frames and unstructured light for one or more frames. In such an embodiment, the structured light may be used to build a three-dimensional (3D) representation of the oral cavity of a patient, including the teeth, gingiva, palate, etc. of the patient. However, any reflective (e.g., shiny) intraoral objects in the patient's oral cavity may not be accurately captured and thus the reflective object may not be included in the 3D representation built from the structured light. For building a 3D representation of the reflective object, the operations of method 400 may be performed.

In some embodiments, the intraoral scanner includes multiple cameras (e.g., four or more cameras). Two or more of the cameras may capture (e.g., may simultaneously capture) images of the reflective object. Each of the cameras may have a known relative position and orientation relative to the light projector and relative to one another. Multiple reflections of the same light projector may also be captured in a same image in some embodiments when the surface of the reflective intraoral object is not flat. However, for a flat or slightly convex surface, at most one specular reflection of light from the light projector will be captured by each camera and represented in a respective image.

At block 410, processing logic identifies a virtual image of the light projector in the one or more images. When light from the light projector is reflected from the reflective intraoral object, a virtual image of the light projector (e.g., a reflection of the light projector) will be captured in the image.

At block 412, processing logic receives an identification of the reflective intraoral object. For example, a doctor may select the reflective intraoral object via a user interface (e.g., a graphical user interface), such as from a drop down menu. At block 414, processing logic may receive an identification of the reflective intraoral object from a reference library based on the received identification. In some embodiments, the operations of block 410 and 412 may be performed prior to receiving the intraoral scan data. Based on the received identification, processing logic may perform a lookup in the reference library for a shape of the reflective intraoral object. In some embodiments, the operations of blocks 410-412 are replaced by processing the image(s) with a trained AI model or standard image processing algorithms that output an estimate of a shape of the reflective intraoral object. In some embodiments, the AI model or image processing algorithms output an estimated shape, which is used to identify a match to a reference object in the reference library.

At block 415, processing logic may determine a position and/or angle of a surface of the reflective object based at least in part on the virtual image of the light projector identified in the image(s) and on the received properties (e.g., shape, dimensions, etc.) of the reflective intraoral object. When the surface of the reflective intraoral object is not flat, each image may create a set of limits on the surface. Based on the received properties of the reflective intraoral object (e.g., the shape and dimensions of the reflective intraoral object), processing logic may solve for the position and orientation of the virtual image (and ultimately of the surface of the reflective object) in six degrees of freedom. Processing logic may minimize a solution that agrees with all of the virtual images that are captured. As the number of virtual images of the light projector increases, the solution increases with accuracy, as each virtual image in each image provides additional constraints on the position of the virtual image. Determining the position of the virtual image in 3D space may include triangulating a position of the virtual image of the light projector between multiple images captured simultaneously by different cameras of the intraoral scanner based on known relative positions and orientations of the light projector and the different cameras and the known properties (e.g., shape and dimensions) of the reflective intraoral object. This may further include solving a correspondence problem that determines which light projectors correspond to which virtual images of light projectors. The 3D shape of the reflective object may help to solve the correspondence problem. Processing logic may then determine a plane that is equidistant between the light projector and the virtual image, and determine the position and angle of the surface of the reflective object based on the position and/or angle of the determined plane, as described previously.

At block 420, processing logic generates an initial 3D surface, optionally with boundaries or ranges around one or more points on the surface of the reflective object (e.g., reflecting an uncertainty around a position of the point on the reflective intraoral object and/or on an uncertainty around an angle of the surface of the reflective intraoral object at the point). As more images that include virtual images of the light projector are captured, the uncertainty with respect to the position and/or angle of points on the surface of the reflective intraoral surface may be decreased based on refining the solved position and orientation (e.g., angle) in six degrees of freedom from other images.

As discussed above, the intraoral scanner may alternate between structured light projection and nonstructured light projection, and the nonstructured light projection may be used in embodiments to determine the shape, position and/or angle of the surface of the reflective intraoral object. In other embodiments, structured light projection may be used to determine the shape, position and/or angle of the surface of the reflective intraoral object using the same techniques that are described above. For example, some structured light projection techniques may project enough rays of light to enable the techniques described herein.

In embodiments, any of methods 300-348 may be used to further increase an accuracy of the surface of the reflective intraoral object.

FIG. 4B illustrates a flow diagram for a method 430 of determining a shape of an unknown reflective intraoral object, in accordance with embodiments of the present disclosure. Method 430 may be performed, for example, when a match to a reference object in a library cannot be found for a reflective intraoral object. For example, method 430 may be performed for nonstandard reflective intraoral objects, such as metal (e.g., gold or silver) fillings. In some embodiments, method 430 is performed at block 315 of method 300.

At block 435 of method 430, processing logic receives first intraoral scan data comprising first images of regions around a reflective intraoral object generated during projection of structured light onto the reflective object by a structured light projector of an intraoral scanner. At block 440, processing logic determines a 3D surface of the regions around the reflective intraoral object based on the first intraoral scan data using the techniques described herein. At block 445, processing logic determines a position of the intraoral scanner relative to the reflective intraoral object based on the 3D surface of the regions around the reflective intraoral object (e.g., since the distance between the intraoral scanner and points on the 3D surface is known).

At block 450, processing logic receives second intraoral scan data comprising images of the reflective intraoral object captured during projection of nonstructured light by multiple nonstructured light projectors. In some embodiments, the intraoral scanner alternates between projection of structured light and nonstructured light. For example, the intraoral scanner may alternate between projection of structured light for one or more frames and non-structured light for one or more frames.

In some embodiments, the intraoral scanner includes multiple cameras (e.g., four or more cameras). Two or more of the cameras may capture (e.g., may simultaneously capture) images of the reflective object. Each of the cameras may have a known position and orientation relative to the light projector and relative to one another. Multiple reflections of the same light projector may also be captured in a same image in some embodiments when the surface of the reflective intraoral object is not flat. However, for a flat or slightly convex surface, at most one specular reflection of light from the light projector will be captured by each camera and represented in a respective image.

At block 455, processing logic identifies virtual images of the nonstructured light projectors in the one or more images.

At block 460, processing logic may estimate solutions to a correspondence problem to determine which virtual images correspond to which nonstructured light projector. In embodiments, solving the correspondence problem without knowledge of the shape of the reflective intraoral object can be challenging. Also, for every nonstructured light projector there may be more then one virtual image (e.g., reflection) of a same nonstructured light projector in an image. Accordingly, the correspondence problem may be a one to many correspondence problem. In some embodiments, the nonstructured light projectors have variations in shape, size and/or color. These variations may amount to coding of slight differences between the light projectors. These variations may make it easier to solve the correspondence problem, as shape, color, size, etc. of virtual images may be taken into account. For example, virtual images may be compared only to light projectors that share a shape and/or color with the virtual images.

In some embodiments, a rough approximation of the surface of the reflective intraoral object may be used to estimate the correspondence.

In some embodiments, the rough estimation is determined using a trained AI model such as a neural network (e.g., a deep neural network (DNN) that receives the one or more images as an input and outputs a suggested surface. The trained AI model may generate surface shape estimates from multiple different images of the reflective intraoral object. These generated surface shape estimates may be averaged to generate a more accurate estimate of the surface shape.

In some embodiments, an estimation of surface normals to the surface of the reflective intraoral object may be determined using a trained AI model such as a neural network (e.g., a deep neural network (DNN)) that receives the one or more images as an input and outputs one or more surface normals to the unknown surface of the reflective intraoral object. The trained AI model may generate surface normal estimates from multiple different images of the reflective intraoral object. In embodiments, the surface shape estimates and surface normal estimates may be used in combination to generate an estimate of the shape of the reflective object's surface that has improved accuracy.

In some embodiments, processing logic tracks virtual images (e.g., 3D positions of virtual images) of the light projectors across sequential images to improve an accuracy of solutions to the correspondence problem. The tracking may be performed as described in U.S. Pat. No. 11,563,929. If a solution to the correspondence problem yields a virtual image having a position that does not correlate to a tracked moving virtual image from one or more preceding or following frames, then the solution to the correspondence problem may be removed from consideration in embodiments. In embodiments, such tracking may also be used in combination with any of the above described methods for determining information about a surface of a reflective object (e.g., methods 200, 240, etc.).

In some instances, a few options for solutions to the correspondence problem still remain (e.g., even after performing one or more of the above operations). In such embodiments, processing logic may perform surface creation for each of the options, and may determine which of the generated surfaces is more reasonable at a later stage (e.g., after additional intraoral scan data is captured). At this time the options determined to be no longer viable may be discarded.

Based on the solution to the correspondence problem, processing logic may determine a position in 3D space of the virtual images of the nonstructured light projectors. The 3D positions of the virtual images may then be used to estimate points on a surface of the reflective intraoral object.

At block 465, processing logic may then estimate a 3D surface of the reflective intraoral object based on the determined solutions to the correspondence problem (and based on the determined points in 3D space determined based on the solution to the correspondence problem).

In some embodiments, an initial surface estimation can be done using a trained AI model (e.g., a NN) on the images, or can be done by computing a smooth surface smoothly connected to the surrounding known surface (e.g., as determined at blocks 435-445). The points at the perimeter of the reflective object (e.g., of non-reflective or partially diffuse objects that border the reflective intraoral object) may provide boundary conditions for the surface of the reflective intraoral object. In some embodiments, the surface of the reflective intraoral object can be represented as a polynomial function (e.g., in some polynomial family like Legenere or Jacobu polynomials or fourier harmonics or any other differentiable surface). In some embodiments, multiple different initial surface guesses for the surface of the reflective intraoral object may be used. In embodiments, rather than performing a global optimization, processing logic performs a local optimization using a starting point that is selected in view of one or more of the aforementioned constraints.

In some embodiments, at block 470 processing logic builds a reflection loss function. Based on the calibration information (e.g., knowledge of relative positions and orientations of light projectors and/or cameras), processing logic can define, for each virtual image, a line in space where the camera is seeing the virtual image from. In some embodiments, processing logic applies a reflection loss function to at least one of the position or the angle of the surface of the reflective intraoral object to determine an error between an expected normal for a surface reflection at the position of the surface of the reflective intraoral object and an estimated normal for the surface reflection at the position of the surface of the reflective intraoral object. In some embodiments, the expected normal is determined based on calibration information indicating relative positions and orientations of the light projector and one or more cameras of the intraoral scanner. For each possible surface function, when this line intersects with a surface, its expected normal should enable the reflection of a light projector that produced the light into a camera. At block 475, processing logic may then determine a position, shape and/or angle of the surface of the reflective intraoral object based on the optimized loss function.

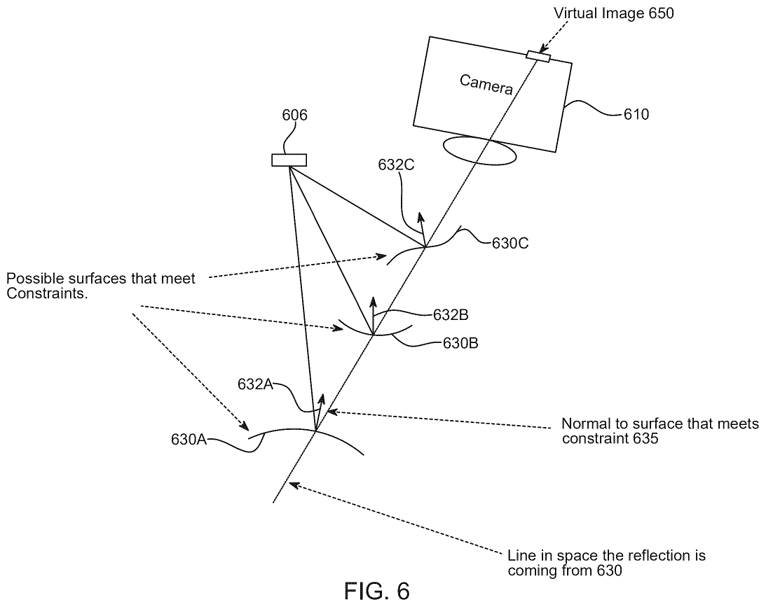

FIG. 6 illustrates multiple possible surfaces of a reflective intraoral object, in accordance with embodiments of the present disclosure. As shown, a light projector 606 may project light onto a reflective object having an unknown surface, and a camera 610 may capture an image of the light projected onto the unknown surface of the reflective object. A specular reflection of the light may be captured by the camera 610, and a virtual image 650 of the light projector 606 may appear in the image due to the specular reflection. The virtual image 650 may be captured by one or more particular pixels of the camera. Since both the shape of the surface of the reflective object and the distance of the reflective object from the intraoral scanner are unknown, there are multiple possibilities for the shape and position (e.g., distance) of the reflective object from the intraoral scanner based on the particular pixels of the camera 610 that captured the virtual image 650.

A virtual line in space 630 may be drawn from the pixels that captured the virtual image to the reflection. Based on determined constraints, multiple different options for positions of the virtual image in 3D space, and positions and angles of the surface of the reflective intraoral object, may be determined. As shown, the possible positions/angles of the surface include surface option 630A, surface option 630B and surface option 630C. For each surface option 630A-C, a different normal to the surface 632A, 632B, 632C may be determined.

In the coordinate system of the intraoral scanner, x and y coordinates may be in a plane along a longitudinal axis of the intraoral scanner. Additionally, depth may correspond to distance from the intraoral scanner along an axis that is normal to the plane and may be represented as z. X and y coordinates may fall on the plane, and the z coordinates may be normal to the plane.

In embodiments, a height of a point (e.g., on the surface of the reflective intraoral object) may be represented by a polynomial, a Fourier, or any other linear function representation, and may be represented as H(x,y).

In embodiments, a normal vector to the surface of the reflective object may be represented using the function N(x,y).

The coordinates x*,y* may represent a point on the surface where the line 630 in space according to where the virtual image is coming from is seen.

A direction from the virtual image to the light projector may be D_{led}, and a direction from the virtual image to the camera may be D_{cam}. The directions D_{led} and D_{cam} may be determined for each point x*,y*.

An expected normal, EN, in x*,y* for the virtual image is the mean/center between D_{led} and D_{cam}.

An error between the expected normal for the virtual image (e.g., reflection) and the surface normal at the point of reflection on the reflective object may be determined according to the function:

Err=∥EN−N(x*,y*)∥2

A loss function may then be computed based on the sum of all of the computed Err values on the surface of the reflective object. Additional components to the loss function can be the edge of the known surface (the regions that were not shiny and could be solved with the structured light 3D mechanism).

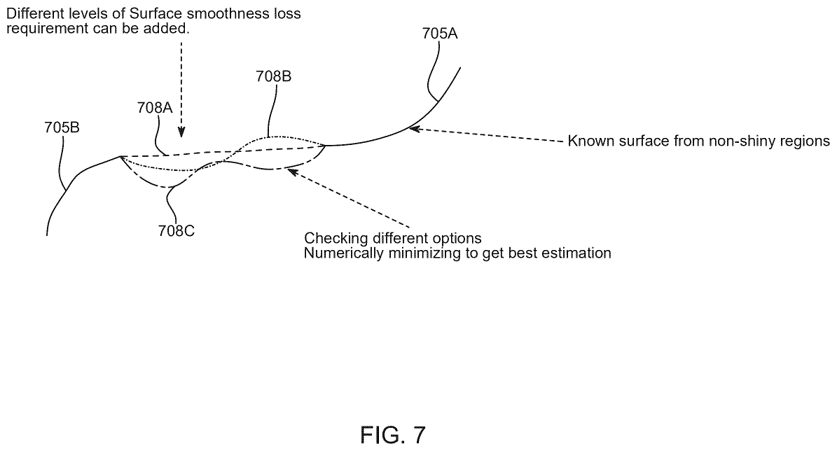

FIG. 7 illustrates multiple possible levels of surface smoothness 708A, 708B, 708C of a reflective intraoral object, in accordance with embodiments of the present disclosure. The boundaries of the reflective object 705A and 705B may be known, and may provide constraints on the surface of the reflective object. The different possible levels of smoothness may be checked to numerically minimize a loss function and produce a best estimation.

Processing logic can run the optimization to minimize the loss by running non-linear optimization on surface function representation parameters. The gradient and/or hessians can be computed by changing the parameters of the loss function and re-evaluating the loss function for each dimension. Alternatively, processing logic can run a derivative computation by method of automatic differentiation. In embodiments, processing logic can add regularization parts to the loss function, penalizing the number of non-zero parameters and/or those having a non-smooth loss.

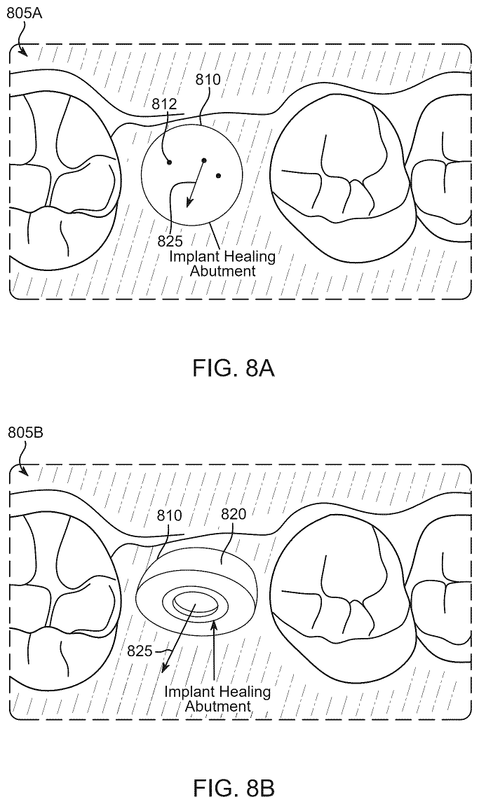

FIG. 8A illustrates view 805A of a portion of an example dental arch with a reflective intraoral object disposed thereon, in accordance with one embodiment of the present disclosure. A boundary 810 of the reflective intraoral object may be determined based on intraoral scan data captured using structured light projection. However, only a few points 812 (and surface angles represented as vectors 825 at those points) may be determined for the surface of the reflective object.

FIG. 8B illustrates the view of FIG. 8A after library data has been used to augment the representation of the reflective intraoral object, in accordance with one embodiment of the present disclosure. As shown, in addition to the boundary 810 of the reflective object, a full 3D surface 820 of the reflective object may be added to the 3D model of the dental arch. A vector 825 showing an angle of the reflective object may match the vector 825 of one or more identified points in FIG. 8A in embodiments.

FIG. 9 illustrates one embodiment of a system 900 for performing intraoral scanning and/or generating a virtual three-dimensional (3D) model of a dental site (e.g., that may include a reflective object). In one embodiment, system 900 carries out one or more operations of the above methods described with reference to FIGS. 2A-4B. System 900 includes a computing device 905 that may be coupled to a scanner 950 and/or a data store 910.

Computing device 905 may include a processing device, memory, secondary storage, one or more input devices (e.g., such as a keyboard, mouse, tablet, and so on), one or more output devices (e.g., a display, a printer, etc.), and/or other hardware components. Computing device 905 may be connected to a data store 910 either directly or via a network. The network may be a local area network (LAN), a public wide area network (WAN) (e.g., the Internet), a private WAN (e.g., an intranet), or a combination thereof. The computing device 905 and/or the data store 910 may be integrated into the scanner 950 in some embodiments to improve performance and mobility.

Data store 910 may be an internal data store, or an external data store that is connected to computing device 905 directly or via a network. Examples of network data stores include a storage area network (SAN), a network attached storage (NAS), and a storage service provided by a cloud computing service provider. Data store 910 may include a file system, a database, or other data storage arrangement.

In some embodiments, a scanner 950 for obtaining three-dimensional (3D) data of a dental site in a patient's oral cavity (referred to as an intraoral scanner) is also operatively connected to the computing device 905. Scanner 950 may include a probe (e.g., a hand held probe) for optically capturing three dimensional structures (e.g., by confocal focusing of an array of light beams).

The scanner 950 may be used to perform an intraoral scan of a patient's oral cavity. A result of the intraoral scan may be a sequence of intraoral scans, each of which may include or be based on multiple images, each captured by a different camera of the intraoral scanner simultaneously or approximately simultaneously. An operator may start recording the video (e.g., sequence of intraoral scans) with the scanner 950 at a first position in the oral cavity, move the scanner 950 within the oral cavity to a second position while the video is being taken, and then stop recording the video. In some embodiments, recording may start automatically as the scanner identifies that it has been inserted into a patient oral cavity. The scanner 950 may transmit the intraoral images or intraoral scans (e.g., 3D point clouds generated from the intraoral images to the computing device 905). Note that in some embodiments the computing device may be integrated into the scanner 950. Computing device 905 may store the image data 935 in data store 910. Alternatively, scanner 950 may be connected to another system that stores the image data in data store 910. In such an embodiment, scanner 950 may not be connected to computing device 905.

In some embodiments, the scanner 950 includes an elongate wand including a probe at a distal end of the wand; a rigid structure disposed within a distal end of the probe; one or more structured light projectors coupled to the rigid structure and one or more non-structured light projectors coupled to the rigid structure, such as non-coherent light projectors and/or near-infrared light projectors; and one or more cameras coupled to the rigid structure. In some applications, each light projector may have an angular field of view (AFOI) of 45-120 degrees. Optionally, the one or more light projectors may utilize a laser diode light source, a light emitting diode (LED) light source, and/or other light source. Further, the structured light projector(s) may include a beam shaping optical element. Further still, the structured light projector(s) may include a pattern generating optical element.

The pattern generating optical element may be configured to generate a light pattern such as a distribution of discrete unconnected spots of light, a checkerboard pattern, a grid pattern, a line pattern, or other pattern. The light pattern may be generated at all planes located between specific distances (e.g., 0-30 mm, 0-20 mm etc.) from the pattern generating optical element when the light source (e.g., laser diode) is activated to transmit light through the pattern generating optical element. In some applications, the pattern generating optical element utilizes diffraction and/or refraction to generate the distribution. Optionally, the pattern generating optical element has a light throughput efficiency of at least 90%.

For some applications, the light projectors and/or the cameras are positioned such that one or more light projector and/or one or more cameras faces an object outside of the wand placed in its field of illumination. Optionally, each camera may face an object outside of the wand placed in its field of view. Additionally, or alternatively, one or more light projectors and/or cameras may face a mirror that reflects light to/from an object being scanned. Further, in some applications, at least 20% of the pattern features are in the field of view of at least one of the cameras.

Scanner 950 may drive each one of one or more structured light projectors to project a light pattern (e.g., a distribution of discrete unconnected spots of light, a checkerboard pattern, etc.) on an intraoral three-dimensional surface and/or may drive one or more non-structured light projectors to project non-structured light onto the intraoral 3D surface. Scanner 950 may further drive each one of one or more cameras to capture an image, the image including one or more image features corresponding to pattern features projected by one of the structured light projectors of the scanner 1050 if structured light was projected. Each one of the one or more cameras may include a camera sensor including an array of pixels. The images captured together at a particular time may constitute an image set and may together form an intraoral scan. The intraoral scans may be transmitted to computing device 1005 and/or stored in data store 1010 as scan data 1035.

Computing device 905 may include an intraoral scan application 910 for facilitating intraoral scanning and generating 3D models of dental arches and/or intraoral objects from intraoral scans. Intraoral scan application 910 may include a reflective object identifier 915, a structured light correspondence solver 918, and/or a model generation module 925. The modules 915, 918, 925 may be software modules that are components of a single software application (e.g., different libraries of a single application), may be distinct software applications, may be firmware, may be hardware modules, and/or a combination thereof. In some embodiments, one or more of the modules 915, 918, 925, or portions thereof, may execute on scanner 950 in addition to, or instead of, on computing device 905.

Structured light correspondence solver 918 may process image data 935 (e.g., images captured during structured light projection by scanner 950) and compare against known projected structured light patterns to determine which captured structured light features correspond to which projected structured light features. Structured light correspondence solver 918 may determine 3D points in space corresponding to the solutions to the correspondence problem, which may correspond to points on a surface of an imaged object. 3D point clouds may be generated from the determined 3D points in space. In embodiments, structured light correspondence solver 918 solves a correspondence problem for sets of images captured at a same time by different cameras of the scanner 950 to generate a 3D point cloud. The 3D point clouds may then be used by model generation module 925 to generate a 3D surface and/or a 3D model of a dental site (e.g., of a dental arch, a portion of a dental arch, etc.).

In embodiments, structured light correspondence solver 918 may execute a correspondence algorithm on intraoral scans to determine the depths of spots or points in the intraoral scans. The structured light correspondence solver 918 may access stored calibration data indicating (a) a camera ray of image feature corresponding to each pixel on the camera sensor of each one of the one or more cameras, and (b) a projector ray corresponding to each of the projected pattern features from each one of the one or more projectors and/or one or more projector rays corresponding to each nonstructured light projector, where each projector ray corresponds to a respective path of pixels on at least one of the camera sensors. Using the calibration data and the correspondence algorithm, structured light correspondence solver 918 may, (1) for each projector ray i, identify for each detected image feature j on a camera sensor path corresponding to ray i, how many other cameras, on their respective camera sensor paths corresponding to ray i, detected respective image features k corresponding to respective camera rays that intersect ray i and the camera ray corresponding to detected image feature j. Ray i is identified as the specific projector ray that produced a detected image feature j for which the highest number of other cameras detected respective image features k. Structured light correspondence solver 918 may further (2) compute a respective three-dimensional position on an intraoral three-dimensional surface at the intersection of projector ray i and the respective camera rays corresponding to the detected image feature j and the respective detected image features k. For some applications, running the correspondence algorithm further includes, following operation (1), removing from consideration projector ray i, and the respective camera rays corresponding to the detected image feature j and the respective detected image features k, and running the correspondence algorithm again for a next projector ray i. Similar correspondence algorithms may also be solved to identify which virtual images of light projectors correspond to which light projectors in embodiments.

Reflective object identifier 915 may analyze received image data 935 (e.g., images captured during nonstructured light projection) to determine position and/or angle of a surface of a reflective object and/or to determine a shape of the surface of the reflective object. In embodiments, reflective object identifier 915 may perform the operations of one or more of methods 200-430.

Model generation module 925 may perform surface registration between intraoral scans (e.g., may stitch together the intraoral scans as discussed above). Model generation module 925 may then generate a virtual 3D model of a dental arch from the registered intraoral scans, as discussed above.

In some embodiments, the data store 910 may include a reference library that includes entries for one or more reference objects 930. The reference objects 930 may include well known equipment that is standard in dentist offices and/or orthodontist offices. Such reference objects 930 may be default reference objects that the data store 910 is prepopulated with. Some reference objects 930 may be custom reference objects that may have been generated by a dental practitioner using a training mode. Each entry for a reference object may include one or more physical properties of that reference object, such as a shape (e.g., a 3D virtual model of the reference object), color, texture, surface pattern, and so on.

A user interface 940 may function during an intraoral scan session. As scanner 950 generates intraoral images and those intraoral images are processed by reflective object identifier 915 and/or model generation module 925, a view screen showing a two-dimensional (2D) or 3D representation of a scanned dental site may be output to a display (e.g., of computing device 905). Images may be stitched together during the intraoral scan session to continuously update the representation of the dental site during the intraoral scan session. Image registration processes and model generation processes may be performed to continually update a 3D surface. The representation of the dental site may show a user of the scanner 950 what areas have been sufficiently imaged and which areas would benefit from further imaging.

In embodiments, one or more AI models (e.g., machine learning models) are used by the intraoral scan application 910 (e.g., by reflective object identifier 915, structured light correspondence solver 918 and/or model generation module 925) to perform one or more operations, as described above. The AI models may be trained to perform, for example, detection and classification of virtual images, estimation of a 3D surface of a reflective intraoral object, and so on. One type of machine learning model that may be used to perform some or all of the above asks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

For model training, a training dataset containing hundreds, thousands, tens of thousands, hundreds of thousands or more intraoral scans, images and/or 3D models should be used to form a training dataset. In embodiments, up to millions of cases of patient dentition that may have underwent a prosthodontic procedure and/or an orthodontic procedure may be available for forming a training dataset, where each case may include various labels of one or more types of useful information. Each case may include, for example, data showing a 3D model, intraoral scans, height maps, color images, NIRI images, etc. of one or more dental sites, data showing pixel-level segmentation of the data (e.g., 3D model, intraoral scans, height maps, color images, NIRI images, etc.) into various dental classes (e.g., tooth, restorative object, gingiva, moving tissue, upper palate, virtual image, reflective object, healing abutment, etc.), data showing one or more assigned classifications for the data, and so on. This data may be processed to generate one or multiple training datasets for training of one or more machine learning models.

In one embodiment, generating one or more training datasets includes gathering one or more intraoral scans with labels. The labels may identify locations of virtual images of a light projector, a 3D shape of a reflective object, and so on. Processing logic may gather a training dataset comprising 2D or 3D images, intraoral scans, 3D surfaces, 3D models, height maps, etc. of dental sites (e.g., of teeth) having one or more associated labels (e.g., pixel-level labeled classes in the form of maps (e.g., probability maps) in embodiments. One or more images, scans, surfaces, and/or models and optionally associated probability maps in the training dataset may be resized in embodiments. For example, a machine learning model may be usable for images having certain pixel size ranges, and one or more image may be resized if they fall outside of those pixel size ranges. The images may be resized, for example, using methods such as nearest-neighbor interpolation or box sampling. The training dataset may additionally or alternatively be augmented. Training of large-scale neural networks generally uses tens of thousands of images, which are not easy to acquire in many real-world applications. Data augmentation can be used to artificially increase the effective sample size. Common techniques include random rotation, shifts, shear, flips and so on to existing images to increase the sample size.

To effectuate training, processing logic inputs the training dataset(s) into one or more untrained machine learning models. Prior to inputting a first input into a machine learning model, the machine learning model may be initialized. Processing logic trains the untrained machine learning model(s) based on the training dataset(s) to generate one or more trained machine learning models that perform various operations as set forth above.

Training may be performed by inputting one or more of the images and/scans into the machine learning model one at a time. Each input may include data from an image or intraoral scan in a training data item from the training dataset. The machine learning model processes the input to generate an output. An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. For pixel level segmentation, for each pixel in the image/scan/surface, the final layer applies a probability that the pixel of the image/scan/surface belongs to a first class, a probability that the pixel belongs to a second class, a probability that the pixel belongs to a third class, and/or one or more additional probabilities that the pixel belongs to other classes.

Accordingly, the output may include one or more prediction and/or one or more a probability map. For example, an output probability map may comprise, for each pixel in an input image/scan/surface, a first probability that the pixel belongs to a first dental class, a second probability that the pixel belongs to a second dental class, and so on.

Processing logic may then compare the generated probability map and/or other output to a known probability map and/or label that was included in the training data item. Processing logic determines an error (i.e., a classification error) based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the deep learning model. After one or more rounds of training, processing logic may determine whether a stopping criterion has been met. A stopping criterion may be a target level of accuracy, a target number of processed images from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset may be used to test the model.

Reference is now made to FIG. 10, which is a schematic illustration of an elongate wand 20 for intraoral scanning, in accordance with some applications of the present disclosure. A plurality of light projectors 22 (e.g., including structured light projectors that project visible light, structured light projectors that project infrared light, and/or non-structured light projectors) and a plurality of cameras 24 are coupled to a rigid structure 26 disposed within a probe 28 at a distal end 30 of the wand. In some applications, during an intraoral scan, probe 28 enters the oral cavity of a subject.

In some particular applications of the present disclosure, an apparatus is provided for intraoral scanning (i.e., an intraoral scanner), the apparatus including an elongate wand with a probe at the distal end. During a scan, the probe may be configured to enter the intraoral cavity of a subject. Multiple light projectors (e.g., miniature structured light projectors and/or non-structured light projectors) as well multiple cameras (e.g., miniature cameras) may be coupled to a rigid structure disposed within a distal end of the probe. Each of the light projectors transmits light using a light source, such as a laser diode, light emitting diode (LED), etc. Each of the structured light projectors may be configured to project a pattern of light defined by a plurality of projector rays when the light source is activated. Each camera may be configured to capture a plurality of images that depict at least a portion of a dental object illuminated by the non-structured light and/or the structured light (e.g., a projected pattern of light as projected by the multiple structured light projectors). For structured light projectors, each of the structured light projectors may further include a pattern generating optical element. The pattern generating optical element may utilize diffraction and/or refraction to generate a light pattern (e.g., where coherent light is used). Alternatively, the pattern generating optical element may be a mask that blocks a portion of the light and passes a remainder of the light. The mask can be a static mask or a changing mask such as a digital micro-mirror (DMD), a display, etc. In some applications, the light pattern may be a distribution of discrete unconnected spots of light. In some applications, the light pattern may be a checkerboard pattern. Other light patterns such as grids, lines, regular distributions of polygons, etc. may additionally or alternatively be used. Optionally, the light pattern maintains the distribution of discrete unconnected spots or other pattern features at all planes located up to a threshold distance (e.g., 30 mm, 40 mm, 60 mm, etc.) from the pattern generating optical element, when the light source (e.g., laser diode) is activated to transmit light through the pattern generating optical element. Each of the cameras includes a camera sensor and objective optics including one or more lenses.

A light pattern includes a plurality of pattern features. Typically, a dense light pattern will have dense pattern features and a sparse light pattern will have sparse pattern features. However, in some embodiments a sparse pattern may have dense pattern features. The pattern features of a light pattern may include, for example, the corners of a checkerboard (e.g., for a checkerboard light pattern). In another example, pattern features may be discrete spots of light. When projecting a pattern comprising pattern features onto a surface of a 3D object, acquired images of the object will comprise a plurality of captured image features corresponding to the pattern features. A pattern feature and an image feature may be an individual well-defined location in the image feature or pattern feature. Examples of image features and pattern features include corners, edges, vertices, points, transitions, dots, stripes, and so on.

For some applications, light projectors 22 are positioned within probe 28 such that one or more light projector 22 faces a 3D surface 32A and/or a 3D surface 32B outside of wand 20 that is placed in its field of illumination, as opposed to positioning the light projectors in a proximal end of the wand and illuminating the 3D surface by reflection of light off a mirror and subsequently onto the 3D surface. Similarly, for some applications, cameras 24 are positioned within probe 28 such that each camera 24 faces a 3D surface 32A, 32B outside of wand 20 that is placed in its field of view, as opposed to positioning the cameras in a proximal end of the wand and viewing the 3D surface by reflection of light off a mirror and into the camera. This positioning of the projectors and the cameras within probe 28 enables the scanner to have an overall large field of view while maintaining a low profile probe.

In some applications, a height H1 of probe 28 is less than 15 mm, height H1 of probe 28 being measured from a lower surface 176 (sensing surface), through which reflected light from 3D surface 32A, 32B being scanned enters probe 28, to an upper surface 178 opposite lower surface 176. In some applications, the height H1 is between 10-15 mm.

In embodiments, the light projectors and cameras disposed at a distal end of an intraoral scanner are non-telecentric. A camera may have a predefined field of view (FOV) and/or a predefined angular field of view (AFOV). Similarly, a light projector may have a predefined field of illumination (FOI) and/or a predefined angular field of illumination (AFOI). The field of view (FOV) of a camera in the intraoral scanner may be understood as the extent of the observable world that is seen at any given moment by the camera. The FOV may be reported as an area measure, e.g. an area at a given distance from the camera or at a given distance below the probe of the intraoral scanner. The angular field of view (AFOV) is correlated to the FOV, and the AFOI is correlated to the FOI. However, herein the AFOV and AFOI are expressed as angles and the FOV and FOI are expressed as an area. In embodiments, the light projectors have an AFOI that cause the FOI of the light projectors to become larger with increased distance from the intraoral scanner. Additionally or alternatively, the cameras have an AFOV that cause the FOV of the cameras to become larger with increased distance/depth from the intraoral scanner. The AFOI of the light projectors may cause light patterns projected by the respective light projectors to have different amounts of interference and/or overlap at different depths. Depth as used in this context may refer to a distance between intraoral scanner (e.g., the light projector and/or camera of the intraoral scanner) and an imaged surface along an imaging axis that is orthogonal to a longitudinal axis of the intraoral scanner (e.g., to a longitudinal axis of a probe of the intraoral scanner that contains the cameras and light projectors).

Each camera may be configured to capture a plurality of images that depict at least a portion of the projected pattern(s) of light as projected by the multiple light projectors on an intraoral object. In some applications, the light projectors may have an AFOI of at least 45 degrees. Optionally, the AFOI may be less than 120 degrees. For structured light projectors, each of the structured light projectors (e.g., visible and/or infrared structured light projectors) may further include a pattern generating optical element. The pattern generating optical element may utilize diffraction and/or refraction to generate a light pattern (e.g., where coherent light is used). Alternatively, the pattern generating optical element may be a mask that blocks a portion of the light and passes a remainder of the light. The mask can be a static mask or a changing mask such as a digital micro-mirror (DMD), a display, etc. In some applications, one or more of the light pattern(s) may be a distribution of discrete unconnected spots of light. In some applications, at least one light pattern may be a checkerboard pattern. Other light patterns such as grids, lines, regular distributions of polygons, etc. may additionally or alternatively be used. Optionally, the light pattern maintains the distribution of discrete unconnected spots or other pattern features at all planes located up to a threshold distance (e.g., 30 mm, 40 mm, 60 mm, etc.) from the pattern generating optical element, when the light source (e.g., laser diode) is activated to transmit light through the pattern generating optical element. Each of the cameras includes a camera sensor and objective optics including one or more lenses.

In some applications, the AFOV of each of the cameras may be at least 45 degrees, e.g., at least 80 degrees, e.g., 85 degrees. Optionally, the AFOV of each of the cameras may be less than 120 degrees, e.g., less than 90 degrees. The fields of view of the various cameras may together form a field of view of the intraoral scanner. In any case, the fields of view and/or angular fields of view of the various cameras may be identical or non-identical. Similarly, the focal length of the various cameras may be identical or non-identical. Further, each camera may be configured to focus at an object focal plane that is located up to a threshold distance from the respective camera sensor (e.g., up to a distance of 10 mm, 20 mm, 30 mm, 40 mm, 50 mm, 60 mm, 70 mm, 80 mm, etc. from the respective camera sensor). As distances increase, the accuracy of the position of the detected surfaces decreases. In one embodiment, beyond the threshold distance the accuracy is below an accuracy threshold. Similarly, in some applications, the AFOI of each of the light projectors (e.g., structured light projectors and/or non-structured light projectors) may be at least 45 degrees and optionally less than 120 degrees. A large field of view (FOV) of the intraoral scanner achieved by combining the respective fields of view of all the cameras may improve accuracy (as compared to traditional scanners that typically have a FOV of 10-20 mm in the x-axis and γ-axis and a depth of capture of about 0-15 or 025 mm) due to reduced amount of image stitching errors, especially in edentulous regions, where the gum surface is smooth and there may be fewer clear high resolution 3-D features. Having a larger FOV for the intraoral scanner enables large smooth features, such as the overall curve of the tooth, to appear in each image frame, which improves the accuracy of stitching respective surfaces obtained from multiple such image frames.

In some applications, the total combined FOV of the various cameras (e.g., of the intraoral scanner) is between about 20 mm and about 50 mm along the longitudinal axis of the elongate wand, and about 20-60 mm (or 20-40 mm) in the z-axis, where the z-axis may correspond to depth. In further applications, the field of view may be about 20 mm, about 25 mm, about 30 mm, about 35 mm, or about 40 mm along the longitudinal axis and/or at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, or at least 80 mm in the z-axis. In some embodiments, the combined field of view may change with depth (e.g., with scanning distance). For example, at a scanning distance of about 4 mm the field of view may be about 20 mm along the longitudinal axis, and at a scanning distance of about 20-50 mm the field of view may be about 30 mm or less along the longitudinal axis. If most of the motion of the intraoral scanner is done relative to the long axis (e.g., longitudinal axis) of the scanner, then overlap between scans can be substantial. In some applications, the field of view of the combined cameras is not continuous. For example, the intraoral scanner may have a first field of view separated from a second field of view by a fixed separation. The fixed separation may be, for example, along the longitudinal axis of the elongate wand.

In some embodiments, the large FOV of the intraoral scanner increases an accuracy of the detected depth of 3D surfaces. For example, the accuracy of a depth measurement of a detected 3D surface may be based on the longitudinal distance between two cameras or between a light projector and a camera, which may represent a triangulation baseline distance. In embodiments, cameras and/or light projectors may be spaced apart in a configuration that provides for increased accuracy of depth measurements for 3D surfaces that, for example, have a depth of up to 30 mm, up to 40 mm, 15-25 mm, and so on.

In some applications, cameras 24 each have a large AFOV β (beta) of at least 45 degrees, e.g., at least 70 degrees, e.g., at least 80 degrees, e.g., 85 degrees. In some applications, the field of view may be less than 120 degrees, e.g., less than 100 degrees, e.g., less than 90 degrees. In experiments performed by the inventors, AFOV B (beta) for each camera being between 80 and 90 degrees was found to be particularly useful because it provided a good balance among pixel size, field of view and camera overlap, optical quality, and cost. Cameras 24 may include a camera sensor 58 and objective optics 60 including one or more lenses. To enable close focus imaging cameras 24 may focus at an object focal plane 50 that is located between 1 mm and 30 mm, e.g., between 4 mm and 24 mm, e.g., between 5 mm and 11 mm, e.g., 9 mm-10 mm, from the lens that is farthest from the camera sensor. Cameras 24 may also detect 3D surfaces located at greater distances from the camera sensor, such as 3D surfaces at 40 mm, 50 mm, 60 mm, 70 mm, 80 mm, 90 mm, and so on from the camera sensor.

As described hereinabove, a large field of view achieved by combining the respective fields of view of all the cameras may improve accuracy due to reduced amount of image stitching errors, especially in edentulous regions, where the gum surface is smooth and there may be fewer clear high resolution 3-D features. Having a larger field of view enables large smooth features, such as the overall curve of the tooth, to appear in each image frame, which improves the accuracy of stitching respective surfaces obtained from multiple such image frames.

Similarly, light projectors 22 may each have a large AFOI a (alpha) of at least 45 degrees, e.g., at least 70 degrees. In some applications, AFOI a (alpha) may be less than 120 degrees, e.g., than 100 degrees.

For some applications, in order to improve image capture, each camera 24 has a plurality of discrete preset focus positions, in each focus position the camera focusing at a respective object focal plane 50. Each of cameras 24 may include an autofocus actuator that selects a focus position from the discrete preset focus positions in order to improve a given image capture. Additionally or alternatively, each camera 24 includes an optical aperture phase mask that extends a depth of focus of the camera, such that images formed by each camera are maintained focused over all 3D surface distances located between 1 mm and 30 mm, e.g., between 4 mm and 24 mm, e.g., between 5 mm and 11 mm, e.g., 9 mm-10 mm, from the lens that is farthest from the camera sensor. In further embodiments, images formed by one or more cameras may additionally be maintained focused over greater 3D surface distances, such as distances up to 40 mm, up to 50 mm, up to 60 mm, up to 70 mm, up to 80 mm, or up to 90 mm.

In some applications, light projectors 22 and cameras 24 are coupled to rigid structure 26 in a closely packed and/or alternating fashion, such that (a) a substantial part of each camera's field of view overlaps the field of view of neighboring cameras, and (b) a substantial part of each camera's field of view overlaps the field of illumination of neighboring projectors. Optionally, at least 20%, e.g., at least 50%, e.g., at least 75% of the projected pattern of light are in the field of view of at least one of the cameras at an object focal plane 50 that is located at least 4 mm from the lens that is farthest from the camera sensor. Due to different possible configurations of the projectors and cameras, some of the projected pattern may never be seen in the field of view of any of the cameras, and some of the projected pattern may be blocked from view by 3D surface 32A, 32B as the scanner is moved around during a scan.

Rigid structure 26 may be a non-flexible structure to which light projectors 22 and cameras 24 are coupled so as to provide structural stability to the optics within probe 28. Coupling all the projectors and all the cameras to a common rigid structure helps maintain geometric integrity of the optics of each light projector 22 and each camera 24 under varying ambient conditions, e.g., under mechanical stress as may be induced by the subject's mouth. Additionally, rigid structure 26 helps maintain stable structural integrity and positioning of light projectors 22 and cameras 24 with respect to each other. As further described hereinbelow, controlling the temperature of rigid structure 26 may help enable maintaining geometrical integrity of the optics through a large range of ambient temperatures as probe 28 enters and exits a subject's oral cavity or as the subject breathes during a scan.

As shown, 3D surface 32A and 3D surface 32B are in a FOV of the probe 28, with 3D surface 32A being relatively close to the probe 28 and 3D surface 32B being relatively far from the probe 28.

Whether a pair of cameras or a pair of a camera and a light projector are used, the accuracy of the triangulation used to determine the depth of 3D surfaces may be roughly estimated by the following equation:

$${z}_{e r r} = \frac{{p}_{e r r} \cdot {z}^{2}}{f \cdot b}$$

Where zₑᵣᵣ is the error in the depth, pₑᵣᵣ is the basic image processing error (generally a sub-pixel error), z is the depth, f is the focal length of the lens, and b is the base line (the distance between two cameras when using stereo imaging or the distance between the camera and the light projector when using structured light). In embodiments, the probe of the intraoral scanner is configured such that the maximum baseline between two cameras or between a camera and a light projector is large and provides a high level of accuracy for triangulation.

In some embodiments, wand 20 corresponds to the intraoral scanner of U.S. Pat. No. 11,896,461, issued Feb. 13, 2024, which is incorporated by reference herein.

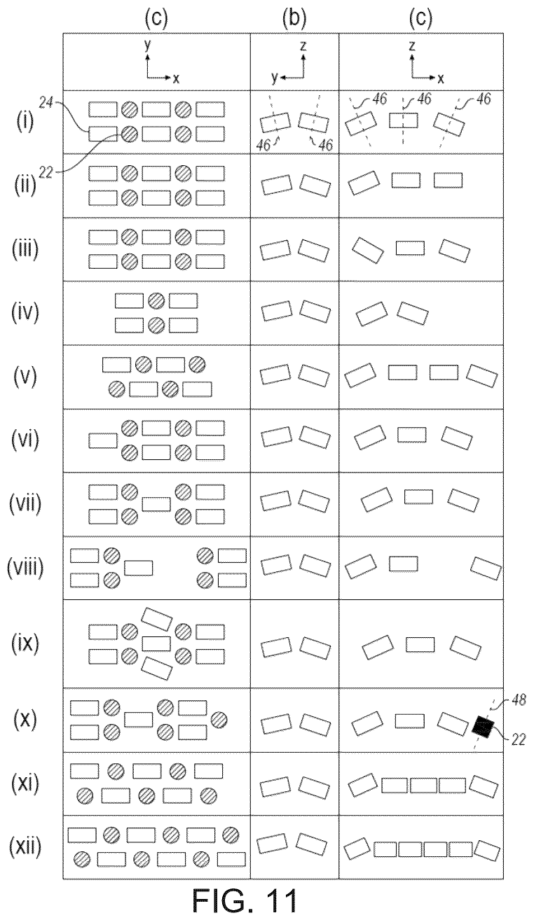

Reference is now made to FIG. 11, which is a chart depicting a plurality of different configurations for the position of light projectors 22 and cameras 24 in probe 28, in accordance with some applications of the present disclosure. Light projectors 22 are represented in FIG. 11 by circles and cameras 24 are represented in FIG. 11 by rectangles. Light projectors may be infrared light projectors, visible light projectors, or a combination thereof. It is noted that rectangles are used to represent the cameras, since typically, each camera sensor 58 and the AFOV β (beta) of each camera 24 have aspect ratios of 1:2. Column (a) of FIG. 11 shows a bird's eye view of the various configurations of light projectors 22 and cameras 24. The x-axis as labeled in the first row of column (a) corresponds to a central longitudinal axis of probe 28. Column (b) shows a side view of cameras 24 from the various configurations as viewed from a line of sight that is coaxial with the central longitudinal axis of probe 28. Column (b) of FIG. 11 shows cameras 24 positioned so as to have optical axes 46 at an angle of 90 degrees or less, e.g., 35 degrees or less, with respect to each other. Column (c) shows a side view of cameras 24 of the various configurations as viewed from a line of sight that is perpendicular to the central longitudinal axis of probe 28.

In one embodiment, the distal-most (toward the positive x-direction in FIG. 11) and proximal-most (toward the negative x-direction in FIG. 11) cameras 24 are positioned such that their optical axes 46 are slightly turned inwards, e.g., at an angle of 90 degrees or less, e.g., 35 degrees or less, with respect to the next closest camera 24. The camera(s) 24 that are more centrally positioned, i.e., not the distal-most camera 24 nor proximal-most camera 24, are positioned so as to face directly out of the probe, their optical axes 46 being substantially perpendicular to the central longitudinal axis of probe 28. It is noted that in row (xi) a projector 22 is positioned in the distal-most position of probe 28, and as such the optical axis 48 of that projector 22 points inwards, allowing a larger number of spots 33 projected from that particular projector 22 to be seen by more cameras 24.

In one embodiment, the number of light projectors 22 in probe 28 may range from two, e.g., as shown in row (iv) of FIG. 11, to six, e.g., as shown in row (xii). In one embodiment, the number of cameras 24 in probe 28 may range from four, e.g., as shown in rows (iv) and (v), to seven, e.g., as shown in row (ix) or eight. It is noted that the various configurations shown in FIG. 11 are by way of example and not limitation, and that the scope of the present disclosure includes additional configurations not shown. For example, the scope of the present disclosure includes more than five projectors 22 positioned in probe 28 and more than seven cameras positioned in probe 28.

In an example application, an apparatus for intraoral scanning (e.g., an intraoral scanner) includes an elongate wand comprising a probe at a distal end of the elongate wand, at least two light projectors disposed within the probe, and at least four cameras disposed within the probe. Each light projector may include at least one light source configured to generate light when activated, and a pattern generating optical element that is configured to generate a pattern of light when the light is transmitted through the pattern generating optical element. Each of the at least four cameras may include a camera sensor and one or more lenses, wherein each of the at least four cameras is configured to capture a plurality of images that depict at least a portion of the projected pattern of light on an intraoral surface. In one embodiment, a majority of the at least two light projectors and the at least four cameras may be arranged in at least two rows that are each approximately parallel to a longitudinal axis of the probe, the at least two rows comprising at least a first row and a second row.

In a further application, a distal-most camera along the longitudinal axis and a proximal-most camera along the longitudinal axis of the at least four cameras are positioned such that their optical axes are at an angle of 90 degrees or less with respect to each other from a line of sight that is perpendicular to the longitudinal axis. Cameras in the first row and cameras in the second row may be positioned such that optical axes of the cameras in the first row are at an angle of 90 degrees or less with respect to optical axes of the cameras in the second row from a line of sight that is coaxial with the longitudinal axis of the probe. A remainder of the at least four cameras other than the distal-most camera and the proximal-most camera have optical axes that are substantially parallel to the longitudinal axis of the probe. Each of the at least two rows may include an alternating sequence of light projectors and cameras.

In a further application, the at least four cameras comprise at least five cameras, the at least two light projectors comprise at least five light projectors, a proximal-most component in the first row is a light projector, and a proximal-most component in the second row is a camera.

In a further application, the distal-most camera along the longitudinal axis and the proximal-most camera along the longitudinal axis are positioned such that their optical axes are at an angle of 35 degrees or less with respect to each other from the line of sight that is perpendicular to the longitudinal axis. The cameras in the first row and the cameras in the second row may be positioned such that the optical axes of the cameras in the first row are at an angle of 35 degrees or less with respect to the optical axes of the cameras in the second row from the line of sight that is coaxial with the longitudinal axis of the probe.

In a further application, the at least four cameras may have a combined field of view of about 25-45 mm or about 20-50 mm along the longitudinal axis and a field of view of about 20-40 mm or about 15-80 mm along a z-axis corresponding to distance from the probe. Other FOVs discussed herein may also be provided.

In the examples FIGS. 10-11, cameras and structured light projectors directly face an object that is scanned. However, in other embodiments one or more structured light projectors and/or cameras may face a mirror, which may redirect light rays and returning light rays towards and/or from an object being scanned. In some embodiments, structured light projectors and cameras are arranged into one or more scan units, which may be arranged along the longitudinal axis of the probe. A scan unit may be understood herein as a unit comprising at least light projector and one or more cameras. In some embodiments, each scan unit comprises at least two cameras having at least partly overlapping fields of view along different camera optical axes. In one embodiment, each scan unit comprises at least four cameras having at least partly overlapping fields of view along different camera optical axes. An advantage of having overlapping fields of view of the cameras is an improved accuracy due to a reduced amount of image stitching errors. A further advantage of utilizing multiple cameras, such as two or more cameras, is that the reliability of the determination of 3D points is improved, whereby the accuracy of the generated digital 3D representation is improved. A scan unit may further comprise one or more lenses such as collimation lenses or projection lenses.

In some embodiments, one or more scan units may face an object being scanned and/or one or more scan units may face a mirror. Scan units that face an object being scanned may be referred to as downward looking scan units, and may have an imaging axis that is at an angle (e.g., perpendicular) to a longitudinal axis of a probe. Scan units that face a mirror may be referred to as forward looking scan units, and may have an imaging axis that is approximately parallel to a longitudinal axis of a probe. In some embodiments, all scan units are downward looking. In some embodiments, all scan units are forward looking. In some embodiments, some scan units are downward looking and some scan units are forward looking. In some embodiments, scan units are used as set forth in U.S. Patent Application No. 63/656,524, filed Jun. 5, 2024, which is incorporated by reference herein in its entirety.

In some embodiments, the intraoral scanner comprises a plurality of scan units positioned along the longitudinal axis of the intraoral scanner. For example, two scan units may be positioned in series along the longitudinal axis of the intraoral scanner. A scan unit may have a predefined field of view (FOV) and/or a predefined angular field of view (AFOV). In some embodiments, the FOV of each scan unit is at least 300 mm², preferably at least 400 mm², preferably at least 500 mm², preferably at least 550 mm², wherein the area is measured for a given predefined focus distance or working distance. As an example, the field of view of the intraoral scanner may be at least 20×20 mm², such as at least 23×23 mm², at a working distance of between 18 mm to 36 mm, such as approximately 20 mm to 24 mm. For some applications, e.g. for dental scanning applications, the intraoral scanner has a working distance of between 10 mm and 100 mm. In some embodiments, a working distance of the projector unit of between 10 mm and 70 mm, such as between 15 mm and 50 mm, is used. Since the scan unit(s) then take up less space inside the intraoral scanner, it also allows for multiple scan units to be placed in succession inside the intraoral scanner. In some embodiments, the intraoral scanner is able to project a pattern in focus at the exit of the tip of the intraoral scanner, e.g. at the optical window of the s intraoral scanner or at an opening in the surface of the intraoral scanner. The working distance may be understood as the object to lens distance where the image is at its sharpest focus. The working distance may also, or alternatively, be understood as the distance from the object to a front lens, e.g. a front lens of the projector unit. The front lens may be the one or more focus lenses of the projector unit.

In some embodiments the elongated probe comprises one or more openings and/or one or more optical windows at a distal end of the elongated probe. In some embodiments, there is an opening and/or an optical window in the elongated probe, wherein the opening/window is associated with each scan unit, such that each scan unit is configured to project the light pattern through said opening or optical window. Accordingly, the elongated probe may comprise an opening and/or an optical window for each scan unit of the intraoral scanner. The optical window may be made in a polymer material such as poly(methyl methacrylate) (PM MA) or in a ceramic or glass material such as Sapphire glass. The optical window may be made in a transparent, crystalline, ceramic material, such as aluminum oxide (Al₂O₃). Alternatively, the optical window may be made of a mineral glass. In some embodiments, the predefined distance mentioned in relation to the FOV of a given scan unit is measured below said opening/window. In some embodiments, the FOV of at least one scan unit is at least 500 mm², such as approximately 23×23 mm², in a distance approximately 0-15 mm, such as 4-7 mm below said opening/window. This may correspond to a working distance of between 15 mm and 50 mm, such as between 15 mm and 36 mm.

The angular field of view (AFOV) of a scan unit may be correlated to the FOV. In accordance with some embodiments, each scan unit defines an angular field of view (AFOV) of between 50° to 80°, such as between 60° to 75°. In accordance with some embodiments, the projector optical axis and the camera optical axis of at least one camera define a camera-projector angle of approximately 5 to 25 degrees, or 5 to 15 degrees, or 5 to 10 degrees, or 8 to 10 degrees. In some embodiments, the scan unit(s) are rotationally symmetric, such that each camera optical axis defines an approximately similar camera-projector angle with the projector unit of the scan unit.

In embodiments, wherein the intraoral scanner comprises at least two scan units, the intraoral scanner has a FOV, which is larger than the FOV of each scan unit. As an example, if the intraoral scanner comprises two scan units, each scan unit having a predefined FOV, then the intraoral scanner will have twice as large a FOV as each scan unit, provided there is no overlap in FOV between the scan units. In some cases, there may be a small overlap in FOV, but in general the intraoral scanner will have a larger field of view, or in other words an extended field of view, compared to one scan unit in isolation. Accordingly, the FOV of the intraoral scanner can be increased/scaled by the number of scan units in the intraoral scanner. In some embodiments, the intraoral scanner comprises at least three scan units. Accordingly, the intraoral scanner may further comprise a third scan unit comprising a third projector unit, said projector unit preferably configured to project the light pattern at a predefined third focus distance. A scan unit may further comprise at least one reflecting element, such as a mirror or a prism, wherein said reflecting element is configured to alter the direction of the light projected by the projector unit of the scan unit. In general, a scan unit can be oriented in many different ways, wherein the orientation is defined according to the projector optical axis of the scan unit. As an example, a scan unit may be oriented such that the projector optical axis of the scan unit is substantially parallel with the longitudinal axis of the intraoral scanner. Additionally, a scan unit may be oriented such that the light from the projector unit is projected in a forward direction, i.e. towards the distal end of the intraoral scanner. In case, a scan unit is oriented in this way, the scan unit is also referred to as being in a forward-looking configuration. In some embodiments, each scan unit comprises a reflecting element. In other embodiments, at least one scan unit comprises a reflecting element.

As another example, a scan unit may be oriented such that the projector optical axis of the scan unit is angled with respect to the longitudinal axis of the intraoral scanner or with respect to a projector optical axis of another scan unit. In some embodiments, at least one of the projector optical axes is substantially orthogonal to the longitudinal axis of the intraoral scanner. Additionally, such a scan unit may be oriented such that the light from the projector unit is projected in a downward direction, i.e. directly towards the dental object to be scanned. In case a scan unit is oriented in this way, the scan unit is also referred to as being in a downward-looking configuration. If the intraoral scanner comprises a plurality of scan units, such as at least two scan units, the scan units may be in different configurations, e.g. a first scan unit in the forward-looking configuration and a second scan unit in the downward-looking configuration. In this embodiment, the first projector optical axis is substantially parallel to the longitudinal axis of the intraoral scanner, and the second projector optical axis is substantially orthogonal to the longitudinal axis of the intraoral scanner. In other words, a first projector optical axis of a first projector unit of the first scan unit is angled with respect to a second projector optical axis of a second projector unit of the second scan unit. This angle may be at least 45°, such as at least 75°, or at least 85°, or approximately 90°.

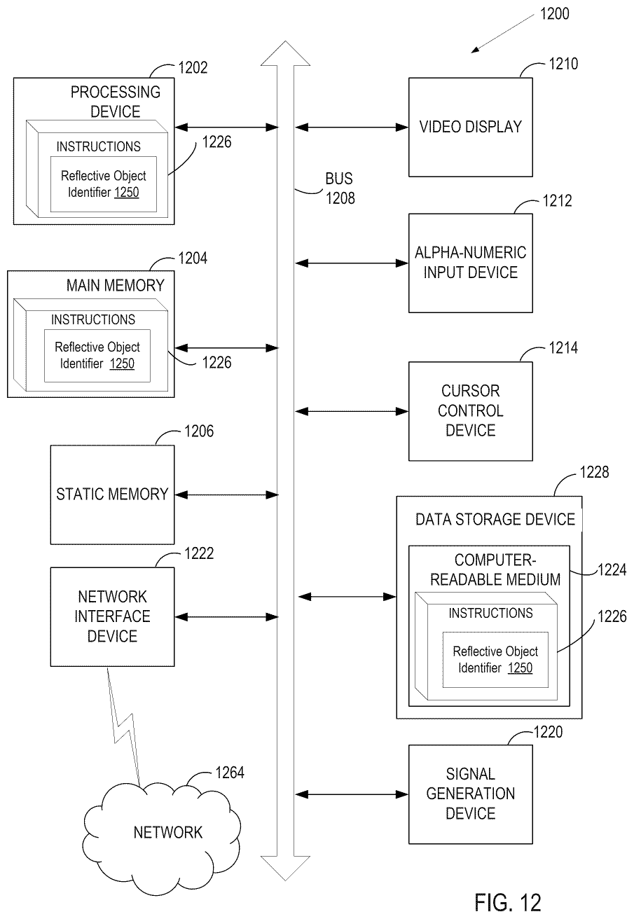

FIG. 12 illustrates a diagrammatic representation of a machine in the example form of a computing device 1200 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 1200 includes a processing device 1202, a main memory 1204 (e.g., read-only memory (ROM), flash memory, dynamic random access memory (DRAM) such as synchronous DRAM (SDRAM), etc.), a static memory 1206 (e.g., flash memory, static random access memory (SRAM), etc.), and a secondary memory (e.g., a data storage device 1228), which communicate with each other via a bus 1208.

Processing device 1202 represents one or more general-purpose processors such as a microprocessor, central processing unit, or the like. More particularly, the processing device 1202 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1202 may also be one or more special-purpose processing devices such as an application specific integrated circuit (ASIC), a field programmable gate array (FPGA), a digital signal processor (DSP), network processor, or the like. Processing device 1202 is configured to execute the processing logic (instructions 1226) for performing operations and steps discussed herein.

The computing device 1200 may further include a network interface device 1222 for communicating with a network 1264. The computing device 1200 also may include a video display unit 1210 (e.g., a liquid crystal display (LCD) or a cathode ray tube (CRT)), an alphanumeric input device 1212 (e.g., a keyboard), a cursor control device 1214 (e.g., a mouse), and a signal generation device 1220 (e.g., a speaker).

The data storage device 1228 may include a machine-readable storage medium (or more specifically a non-transitory computer-readable storage medium) 1224 on which is stored one or more sets of instructions 1226 embodying any one or more of the methodologies or functions described herein. Wherein a non-transitory storage medium refers to a storage medium other than a carrier wave. The instructions 1226 may also reside, completely or at least partially, within the main memory 1204 and/or within the processing device 1202 during execution thereof by the computer device 1200, the main memory 1204 and the processing device 1202 also constituting computer-readable storage media.

The computer-readable storage medium 1224 may also be used to store a reflective object identifier 1250, which may correspond to similarly named reflective object identifier 915 of FIG. 9. The computer readable storage medium 1224 may also store a software library containing methods that call foreign object identifier 1250. While the computer-readable storage medium 1224 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

Multiple example implementations are now described.

A first example implementation is a method comprising: receiving intraoral scan data generated by an intraoral scanner, the intraoral scan data comprising one or more images of a reflective intraoral object generated during projection of light onto the reflective intraoral object by a light projector of the intraoral scanner; identifying a virtual image of the light projector in the one or more images caused by reflection of the light off of the reflective intraoral object; and determining at least one of a position or an angle of a surface of the reflective intraoral object based at least in part on the virtual image of the light projector identified in the one or more images.

A second example implementation may extend the first example implementation, wherein determining at least one of the position or the angle of the surface of the reflective intraoral object based at least in part on the virtual image of the light projector comprises: determining a position of the virtual image in the one or more images; determining a plane that is equidistant between the light projector and the virtual image of the light projector for the one or more images; and determining at least one of the position or the angle of the surface of the reflective intraoral object based on at least one of a position or an angle of the determined plane.

A third example implementation may extend the second example implementation, wherein a vector normal to the plane is on a straight line that connects the light projector to the virtual image of the light projector.

A fourth example implementation may extend any of the first through third example implementations, wherein the one or more images comprise a plurality of images, each image of the plurality of images captured by a different camera of a plurality of cameras of the intraoral scanner at a same time. The fourth example implementation further comprises triangulating a position of the virtual image of the light projector from the plurality of images using calibration data comprising a known relative position and orientation of the light projector and the plurality of cameras.

A fifth example implementation may extend any of the first through fourth example implementations, wherein the one or more images comprise a plurality of images taken from a plurality of intraoral scanner positions relative to the reflective intraoral object. The fifth example implementation further comprises generating an initial three-dimensional (3D) surface of the reflective intraoral object using at least one of the determined position or the determined angle of the surface of the reflective intraoral object from the plurality of images.

A sixth example implementation may extend the fifth example implementation. The sixth example implementation further comprises determining that the reflective intraoral object corresponds to a reference object having one or more known properties; and modifying at least one of the plurality of images or the initial 3D surface by adding additional data about the reflective intraoral object to at least one of the plurality of images or the initial 3D surface based on the one or more known properties of the reference object.

A seventh example implementation may extend the sixth example implementation. The seventh example implementation further comprises receiving a selection of the reference object from a reference library; and registering the selected reference object with the reflective intraoral object.

An eighth example implementation may extend any of the sixth through seventh example implementations, wherein determining that the reflective intraoral object corresponds to the reference object comprises processing at least one of the one or more images, the 3D surface, or a two-dimensional (2D) projection of the 3D surface using a trained artificial intelligence (AI) model that outputs at least one of a) location information for the reflective intraoral object or b) an identification of the reflective intraoral object as an instance of the reference object.

A ninth example implementation may extend any of the sixth through eighth example implementations. The ninth example implementation further comprises performing image processing on at least one of the one or more images, the 3D surface, or a two-dimensional (2D) projection of the 3D surface to determine a probable object represented in the one or more images, wherein the probable object comprises one or more physical properties; comparing the one or more physical properties of the probable object to the one or more known properties of the reference object; and determining that the one or more physical properties of the probable object match the one or more known properties the reference object as a result of the comparing.

A tenth example implementation may extend the ninth example implementation, wherein the one or more physical properties comprise at least one of a reflectivity, a diffraction, a reactivity to a wavelength of light, a texture, a surface pattern, a color or a shape.

An eleventh example implementation may extend any of the fifth through tenth example implementations, wherein the plurality of images were captured sequentially. The eleventh example implementation further comprises tracking a position of the virtual image of the light projector across the plurality of images.

A twelfth example implementation may extend any of the first through eleventh example implementations, wherein the intraoral scanner comprises a plurality of light projectors that project onto the reflective intraoral object during intraoral scanning. The twelfth example implementation further comprises solving a correspondence problem to determine, for each virtual image in each image of the one or more images, which light projector of the plurality of light projectors corresponds to the virtual image.

A thirteenth example implementation may extend the twelfth example implementation, wherein the one or more images comprise a plurality of images, each image of the plurality of images captured by a different camera of a plurality of cameras of the intraoral scanner at a same time, and wherein solving the correspondence problem comprises: solving the correspondence problem for a light projector of the plurality of light projectors for which a highest number of images of the plurality of images provide a triangulation of a virtual image of the light projector that approximately matches; and subsequently solving the correspondence problem for a next light projector of the plurality of light projectors for which a next highest number of images of the plurality of images provide a triangulation of a virtual image of the next light projector that approximately matches.

A fourteenth example implementation may extend the thirteenth example implementation. The fourteenth example implementation further comprises removing the light projector and the virtual images of the light projector from consideration before solving the correspondence problem for the next light projector.

A fifteenth example implementation may extend any of the twelfth through fourteenth example implementations. The fifteenth example implementation further comprises estimating a shape of the surface of the reflective intraoral object; and using the estimated shape of the surface of the reflective intraoral object in solving the correspondence problem.

A sixteenth example implementation may extend the fifteenth example implementation, wherein the estimated shape of the surface of the reflective intraoral object is determined by processing the one or more images using a trained artificial intelligence (AI) model that outputs the estimated shape of the surface of the reflective intraoral object.

A seventeenth example implementation may extend any of the fifteenth through sixteenth example implementations, wherein the estimated shape of the surface of the reflective intraoral object is determined by: processing the one or more images using a trained artificial intelligence (AI) model that outputs surface normals to the surface of the reflective intraoral object; and using the estimated surface normals to the surface of the reflective intraoral object in solving the correspondence problem.

An eighteenth example implementation may extend any of the first through seventeenth example implementations. The eighteenth example implementation further comprises estimating an accuracy of at least one of the position or the angle of a surface of the reflective intraoral object, wherein the accuracy increases with an increased number of captured images.

A nineteenth example implementation may extend the eighteenth example implementation. The nineteenth example implementation further comprises outputting an indication of the accuracy to a display during intraoral scanning.

A twentieth example implementation may extend the nineteenth example implementation. The twentieth example implementation further comprises determining that the accuracy is below an accuracy threshold; and outputting a notice to capture additional images of the reflective intraoral object.

A twenty-first example implementation may extend any of the first through twentieth example implementations, wherein the reflective intraoral object is partially diffuse. The twenty-first example implementation further comprises determining one or more points on the surface of the reflective intraoral object based on diffused scattered light from an interaction of the light with the reflective intraoral object in the one or more images, wherein a shape of the surface of the reflective intraoral object is determined based on a combination of the diffused scattered light and the reflected light captured in the one or more images.

A twenty-second example implementation may extend any of the first through twenty-first example implementations, wherein at least some of the one or more images show one or more teeth in addition to the reflective intraoral object, and wherein the intraoral scan data further comprises one or more point clouds of at least portions of the one or more teeth determined based on structured light projection. The twenty-second example implementation further comprises stitching the one or more images to the one or more point clouds; and determining a position and orientation of the intraoral scanner relative to the reflective intraoral object and the one or more teeth based on a result of the stitching.

A twenty-third example implementation may extend any of the first through twenty-second example implementations, wherein the one or more images comprise a plurality of images, each image of the plurality of images captured by a different camera of a plurality of cameras of the intraoral scanner at a same time. The twenty-third example implementation further comprises identifying one or more surface features of the reflective intraoral object in the plurality of images; and triangulating positions of the one or more surface features based on known relative positions and orientations of the plurality of cameras.

A twenty-fourth example implementation may extend any of the first through twenty-third example implementations. The twenty-fourth example implementation further comprises determining at least one of a center or one or more corners of the virtual image of the light projector in the one or more images, wherein at least one of the center or the one or more corners of the virtual image is used in determining at least one of the position or the angle of the surface of the reflective intraoral object.

A twenty-fifth example implementation may extend any of the first through twenty-fourth example implementations. The twenty-fifth example implementation further comprises applying a reflection loss function to at least one of the position or the angle of the surface of the reflective intraoral object to determine an error between an expected normal for a surface reflection at the position of the surface of the reflective intraoral object and an estimated normal for the surface reflection at the position of the surface of the reflective intraoral object.

A twenty-sixth example implementation may extend the twenty-fifth example implementation, wherein the expected normal is determined based on calibration information indicating relative positions and orientations of the light projector and one or more cameras of the intraoral scanner.

A twenty-seventh example implementation may extend any of the first through twenty-sixth example implementations, wherein the surface of the reflective intraoral object is not flat.

A twenty-eighth example implementation may extend any of the first through twenty-seventh example implementations, wherein the reflective intraoral object is a healing abutment.

A twenty-ninth example implementation may extend any of the first through twenty-eighth example implementations, wherein the reflective intraoral object is a metal filling.

A thirtieth example implementation may extend any of the first through twenty-ninth example implementations, wherein the light projector is a uniform light projector, and wherein a position and orientation of the uniform light projector relative to a plurality of cameras of the intraoral scanner is known based on a calibration procedure.

A thirty-first example implementation is a non-transitory computer readable medium comprising instructions that, when executed by a processing device, cause the processing device to perform the method of any of the first through thirtieth example implementations.

A thirty-second example implementation is a system comprising: the intraoral scanner configured to generate the intraoral scan data; and a computing device configured to perform the method of any of the first through thirtieth example implementations.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. An intraoral scanning system, comprising:
an intraoral scanner configured to generate intraoral scan data; and
one or more processors configured to:
receive the intraoral scan data generated by the intraoral scanner, the intraoral scan data comprising one or more images of a reflective intraoral object generated during projection of light onto the reflective intraoral object by a light projector of the intraoral scanner;
identify a virtual image of the light projector in the one or more images caused by reflection of the light off of the reflective intraoral object; and
determine at least one of a position or an angle of a surface of the reflective intraoral object based at least in part on the virtual image of the light projector identified in the one or more images.

2. The intraoral scanning system of claim 1, wherein determining at least one of the position or the angle of the surface of the reflective intraoral object based at least in part on the virtual image of the light projector comprises:
determining a position of the virtual image in the one or more images;
determining a plane that is equidistant between the light projector and the virtual image of the light projector for the one or more images; and
determining at least one of the position or the angle of the surface of the reflective intraoral object based on at least one of a position or an angle of the determined plane.

3. The intraoral scanning system of claim 2, wherein a vector normal to the plane is on a straight line that connects the light projector to the virtual image of the light projector.

4. The intraoral scanning system of claim 1, wherein the one or more images comprise a plurality of images, each image of the plurality of images captured by a different camera of a plurality of cameras of the intraoral scanner at a same time, and wherein the one or more processors are further configured to:
triangulate a position of the virtual image of the light projector from the plurality of images using calibration data comprising a known relative position and orientation of the light projector and the plurality of cameras.

5. The intraoral scanning system of claim 1, wherein the one or more images comprise a plurality of images taken from a plurality of intraoral scanner positions relative to the reflective intraoral object, and wherein the one or more processors are further configured to:
generate an initial three-dimensional (3D) surface of the reflective intraoral object using at least one of the determined position or the determined angle of the surface of the reflective intraoral object from the plurality of images.

6. The intraoral scanning system of claim 5, wherein the one or more processors are further configured to:
determine that the reflective intraoral object corresponds to a reference object having one or more known properties; and
modify at least one of the plurality of images or the initial 3D surface by adding additional data about the reflective intraoral object to at least one of the plurality of images or the initial 3D surface based on the one or more known properties of the reference object.

7. The intraoral scanning system of claim 6, wherein the one or more processors are further configured to:
receive a selection of the reference object from a reference library; and
register the selected reference object with the reflective intraoral object.

8. The intraoral scanning system of claim 6, wherein determining that the reflective intraoral object corresponds to the reference object comprises processing at least one of the one or more images, the initial 3D surface, or a two-dimensional (2D) projection of the 3D surface using a trained artificial intelligence (AI) model that outputs at least one of a) location information for the reflective intraoral object or b) an identification of the reflective intraoral object as an instance of the reference object.

9. The intraoral scanning system of claim 6, wherein the one or more processors are further configured to:
perform image processing on at least one of the one or more images, the initial 3D surface, or a two-dimensional (2D) projection of the initial 3D surface to determine a probable object represented in the one or more images, wherein the probable object comprises one or more physical properties;
compare the one or more physical properties of the probable object to the one or more known properties of the reference object; and
determine that the one or more physical properties of the probable object match the one or more known properties the reference object as a result of the comparing, wherein the one or more physical properties comprise at least one of a reflectivity, a diffraction, a reactivity to a wavelength of light, a texture, a surface pattern, a color or a shape.

10. The intraoral scanning system of claim 5, wherein the plurality of images were captured sequentially, and wherein the one or more processors are further configured to:
track a position of the virtual image of the light projector across the plurality of images.

11. The intraoral scanning system of claim 1, wherein the one or more processors are further configured to:
determine at least one of a center or one or more corners of the virtual image of the light projector in the one or more images, wherein at least one of the center or the one or more corners of the virtual image is used in determining at least one of the position or the angle of the surface of the reflective intraoral object.

12. The intraoral scanning system of claim 1, wherein the intraoral scanner comprises a plurality of light projectors that project onto the reflective intraoral object during intraoral scanning, and wherein the one or more processors are further configured to:
solve a correspondence problem to determine, for each virtual image in each image of the one or more images, which light projector of the plurality of light projectors corresponds to the virtual image.

13. The intraoral scanning system of claim 12, wherein the one or more images comprise a plurality of images, each image of the plurality of images captured by a different camera of a plurality of cameras of the intraoral scanner at a same time, and wherein solving the correspondence problem comprises:
solving the correspondence problem for a light projector of the plurality of light projectors for which a highest number of images of the plurality of images provide a triangulation of a virtual image of the light projector that approximately matches; and
subsequently solving the correspondence problem for a next light projector of the plurality of light projectors for which a next highest number of images of the plurality of images provide a triangulation of a virtual image of the next light projector that approximately matches.

14. The intraoral scanning system of claim 12, wherein the one or more processors are further configured to:
estimate a shape of the surface of the reflective intraoral object; and
use the estimated shape of the surface of the reflective intraoral object in solving the correspondence problem.

15. The intraoral scanning system of claim 1, wherein the one or more processors are further configured to:
estimate an accuracy of at least one of the position or the angle of the surface of the reflective intraoral object, wherein the accuracy increases with an increased number of captured images; and
output an indication of the accuracy to a display during intraoral scanning.

16. The intraoral scanning system of claim 15, wherein the one or more processors are further configured to:
determine that the accuracy is below an accuracy threshold; and
output a notice to capture additional images of the reflective intraoral object.

17. The intraoral scanning system of claim 1, wherein the light projector is a uniform light projector, and wherein a position and orientation of the uniform light projector relative to a plurality of cameras of the intraoral scanner is known based on a calibration procedure.

18. The intraoral scanning system of claim 1, wherein the reflective intraoral object is partially diffuse, and wherein the one or more processors are further configured to:
determine one or more points on the surface of the reflective intraoral object based on diffused scattered light from an interaction of the light with the reflective intraoral object in the one or more images, wherein a shape of the surface of the reflective intraoral object is determined based on a combination of the diffused scattered light and the reflected light captured in the one or more images.

19. The intraoral scanning system of claim 1, wherein at least some of the one or more images show one or more teeth in addition to the reflective intraoral object, wherein the intraoral scan data further comprises one or more point clouds of at least portions of the one or more teeth determined based on structured light projection, and wherein the one or more processors are further configured to:
stitch the one or more images to the one or more point clouds; and
determine a position and orientation of the intraoral scanner relative to the reflective intraoral object and the one or more teeth based on a result of the stitching.

20. The intraoral scanning system of claim 1, wherein the one or more images comprise a plurality of images, each image of the plurality of images captured by a different camera of a plurality of cameras of the intraoral scanner at a same time, and wherein the one or more processors are further configured to:
identify one or more surface features of the reflective intraoral object in the plurality of images; and
triangulate positions of the one or more surface features based on known relative positions and orientations of the plurality of cameras.

21. The intraoral scanning system of claim 1, wherein the one or more processors are further configured to:
apply a reflection loss function to at least one of the position or the angle of the surface of the reflective intraoral object to determine an error between an expected normal for a surface reflection at the position of the surface of the reflective intraoral object and an estimated normal for the surface reflection at the position of the surface of the reflective intraoral object.
